SEQUENCE LISTING (17 sequences)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 100 aa, Homo sapiens
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 3 - protein, 22 aa, Unknown
  Description of Unknown APO-AI mimetic peptide 22A
PVLDLFRELLNELLEALKQKLK

SEQ ID NO: 4 - protein, 11 aa, Artificial Sequence
  Description of Artificial Sequence Synthetic peptide
LRLTRKRGLKL

SEQ ID NO: 5 - protein, 13 aa, Artificial Sequence
  Description of Artificial Sequence Synthetic peptide
GTTRLTRKRGLKL

SEQ ID NO: 6 - protein, 18 aa, Artificial Sequence
  Description of Artificial Sequence Synthetic peptide
DWFKAFYDKVAEKFKEAF

SEQ ID NO: 7 - protein, 18 aa, Artificial Sequence
  Description of Artificial Sequence Synthetic peptide
EKLKAKLEELKAKLEELL

SEQ ID NO: 8 - protein, 18 aa, Artificial Sequence
  Description of Artificial Sequence Synthetic peptide
EKLKELLEKLLEKLKELL

SEQ ID NO: 9 - protein, 18 aa, Artificial Sequence
  Description of Artificial Sequence Synthetic peptide
EKLLELLKKLLELLKELL

SEQ ID NO: 10 - protein, 18 aa, Artificial Sequence
  Description of Artificial Sequence Synthetic peptide
EKLKELLEKLLEKLKEKL

SEQ ID NO: 11 - protein, 18 aa, Artificial Sequence
  Description of Artificial Sequence Synthetic peptide
EELKEKLEELKEKLEEKL

SEQ ID NO: 12 - protein, 11 aa, Artificial Sequence
  Description of Artificial Sequence Synthetic peptide
LRLTRKRGLKL

SEQ ID NO: 13 - protein, 12 aa, Artificial Sequence
  Description of Artificial Sequence Synthetic peptide
GTTRLTRKRGKL

SEQ ID NO: 14 - protein, 36 aa, Artificial Sequence
  Description of Artificial Sequence Synthetic polypeptide
EKLKAKLEELKAKLEELLEKLKAKLEELKAKLEELL

SEQ ID NO: 15 - protein, 29 aa, Artificial Sequence
  Description of Artificial Sequence Synthetic peptide
DWFKAFYDKVAEKFKEAFLRLTRKRGLKL

SEQ ID NO: 16 - protein, 29 aa, Artificial Sequence
  Description of Artificial Sequence Synthetic peptide
LRLTRKRGLKLEKLLELLKKLLELLKELL

SEQ ID NO: 17 - protein, 4 aa, Unknown
  Description of Unknown “DEVD” motif peptide
DEVD

(19) United States
(12) Patent Application Publication
(10) Pub. No.: US 2021/0024882 A1
(43) Pub. Date: Jan. 28, 2021

(54) COMPOSITIONS AND METHODS FOR ENHANCING CELL CULTURE

(71) Applicant: LIFE TECHNOLOGIES CORPORATION, Carlsbad, CA (US)

(72) Inventors: Sarya MANSOUR, Grand Island, NY (US); Joanna KERN, Grand Island, NY (US); Anson PIERCE, Amherst, NY (US); Pei-Yi LIN, Amherst, NY (US)

(21) Appl. No.: 16/921,626

(22) Filed: Jul. 6, 2020

Related U.S. Application Data
(60) Provisional application No. 62/871,409, filed on Jul. 8, 2019.

(51) Int. Cl. C12N 5/00; C12N 13/00; C12N 5/0783; C12N 15/87

(52) U.S. Cl. CPC C12N 5/0037; C12N 13/00; C12N 2501/998; C12N 15/87; C12N 5/0636

(57) ABSTRACT

Provided herein are improvements in cell culture methods and compositions related thereto. In partial particular, provided herein are compositions and methods, and kits increasing the cellular division times and viability. Also provided herein are compositions and method for performing electroporation where high levels of electroporation efficiency are achieved and where deleterious effect of electroporation on cells are decreased.

### CROSS-REFERENCE TO RELATED APPLICATIONS

This application claims the benefit of priority to U.S. Provisional Patent Application No. 62/871,409 filed Jul. 8, 2019, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 2, 2020, is named LT01457_SL.txt and is 8,090 bytes in size.

### FIELD OF THE INVENTION

Provided herein are improvements in cell culture methods and compositions related thereto. In partial particular, provided herein are compositions and methods, and kits increasing the cellular division times and viability. Also provided herein are compositions and method for performing electroporation where high levels of electroporation efficiency are achieved and where deleterious effect of electroporation on cells are decreased.

### BACKGROUND

Cell culture compositions and methods are known in the art. In many instances, it is desirable to culture cells under conditions where the cells expand to high numbers and maintain a high number of viable cells in the cell population. Provided herein are compositions and methods directed to achieving these goals, as well as other goals.

When cells are to be used for therapeutic purposes, it is generally desirable to culture these cells in the absence of blood serum. Reasons for this include the possibility that the cells will become contaminated with adventitious agents present in serum (e.g., viruses, prions, Mycoplasma, etc.). Also, even sera pooled from the blood of a substantial number (e.g., 100 or more) animals has substantial lot to lot variability when used in mammalian cell culture (see FIG. 1). As can be seen from the data presented in FIG. 1, different lots of commercially available human serum show substantial variation (15% to 50%) in total T cell yields. Substantial variation as also found with respect to transduction efficiency (data not shown). The above are some reasons why serum replacements are often used.

Electroporation is a method by which material can be introduced into cells. A solution containing cells and the material to be introduced are exposed to a brief high intensity electric field. The electric field “porates” the cells, producing transient pores in their outer membranes, allowing diffusion of the material in the solution into the cells.

One issue with the electroporation of cells is that this process often decreases cells viability. Further, a balance is often sought between the inverse correlation electroporation efficiency and cell viability. Potassium, as an example, in physiological levels equal to intracellular amounts tends to increase viability in electroporated cells (e.g., van den Hoff et al., Nucleic Acids Res., 20:2902 (1992)). The presence of calcium ions have been reported to increase viability of cells following electroporation. It is postulated that the reason for the increase in viability is reported to be a contribution by calcium in the resealing process after electroporation. Table 1 of van den Hoff et al. (Nucleic Acids Res., 20:2902 (1992)) essentially shows that the higher the electrical charge applied to the cells the lower the cell viability, with the highest cell viability measured being around 69%.

Osmolarity of the electroporation medium affects cell viability and the efficiency of movement of large molecules through cell membranes. For example, van den Hoff et al. (Nucleic Acids Res., 18:6464 (1990) recommends against the use of hypotonic electroporation media.

There is a need for electroporation methods that lead to both high levels of introduction of molecules into cells while maintaining high levels of cell viability.

### BRIEF SUMMARY

Provided are compositions and methods for culturing and/or expanding cells (e.g., human cells) with high cell viability. Further provided herein are, inter alia, compositions, methods systems, kits, and methods for the introduction of macromolecules into cells where the cells maintain high viability. Thus, in general, provided herein are compositions and methods that relate to cellular processes for the maintenance of high cell viability and the production of cellular compositions where the cells in the compositions maintain a high level of viability.

In some aspects, provided herein are methods for preparing serum free cell culture media, as well as composition used in such methods and the resulting culture media prepared by such methods. Such methods include those in which one or more lipoprotein particle composition and/or one or more lipoprotein is added to a basal culture medium. In many instances, lipoprotein particle compositions and/or lipoprotein are added in amounts to function as a serum replacement.

Lipoprotein particles used in methods and present in compositions set out here may comprise one or more lipoprotein particle selected from the group consisting of (a) high density lipoprotein particles, (b) low density lipoprotein particles, and (c) very low density lipoprotein particles, as well as other types of lipoprotein particles.

Lipoprotein particles used in methods and present in compositions set out here may be obtained from a natural source (e.g., the blood or a mammal, such as a human) or may be synthetically produced (e.g., synthetic lipoprotein particles, such as synthetic high density lipoprotein particles). In some instances, synthetic lipoprotein particles may comprise Apolipoprotein AI, Apolipoprotein AII, Apolipoprotein IV, Apolipoprotein-CI, Apolipoprotein III, Apolipoprotein D, Apolipoprotein E and/or a portion of one or more of such apolipoproteins.

Further, apolipoproteins present in compositions and used in methods set out herein may be obtained from a natural source (e.g., the blood or a mammal, such as a human) and/or recombinantly produced. Additionally, recombinantly production apolipoproteins and/or portions thereof may performed using a non-mammalian cell (e.g., a bacterial cell, a plant cell, and insect cell, etc.).

In some aspects, provided herein are serum free cell culture media. Such culture media may comprise one or more lipoprotein. Further, such culture media may support the expansion of mammalian cells, wherein the expansion of the mammalian cells is increased by at least 10% (e.g., from about 10% to about 75%, from about 10% to about 70%, from about 10% to about 55%, from about 10% to about 45%, from about 10% to about 35%, from about 10% to about 25%, from about 20% to about 70%, from about 20% to about 55%, etc.) in the serum free cell culture medium comprising the one or more lipoprotein as compared to the same cell expanded in culture medium without the one or more lipoprotein but containing serum. Serum free cell culture medium set out herein may contain one of the one or more lipoprotein compound (e.g., Apolipoprotein AI, Apolipoprotein AII, Apolipoprotein IV, Apolipoprotein-CI, Apolipoprotein III, Apolipoprotein D, and Apolipoprotein E, etc.) and/or one or more sub-portion of a lipoprotein. Further, lipoprotein and/or lipoprotein sub-portions may be components of a lipoprotein particle (e.g., a high density lipoprotein particle, a low density lipoprotein particles, a very low density lipoprotein particles, etc.).

Lipoprotein particle present in culture media set out herein (e.g., serum-free culture media) may be obtained from a natural source (e.g., the blood of a mammal, such as a human) or may be non-naturally occurring (e.g., synthetically produced). Further, non-naturally occurring lipoprotein particles may contain one or more non-naturally occurring protein, one or more naturally occurring apolipoprotein, one or more portion of a naturally occurring apolipoprotein, or one or more combination of these.

Cells that may be cultured using compositions and methods set out herein include mammalian cells, such as hybridoma cells, Chinese Hamster Ovary (CHO) cells, human cells, etc.). Further, such cells may be derived from a particular tissue (e.g., liver, spleen, lymph node, lung, etc.) or be of a cell category type (e.g., immune system cells), and/or a specific cells type (e.g., FoxP3+ regulatory T cells, B cells). Such cells may also be T cells and/or specific T cells such as regulatory T cells (e.g., FoxP3+ regulatory T cells, FoxP3− regulatory T cells, etc.), CD4+ T cells, CD8+ T cells, T_{H}1 cells, T_{H}2 cells, T_{H}3 cells, T_{H}17 cells, T_{H}9 cells, T_{FH} cells, etc.

In some instances, provided herein are method for expanding mammalian cells. Such methods may comprise incubating mammalian cells in serum free cell culture media comprising one or more lipoprotein compound under conditions that allow for expansion of the mammalian cells.

Lipoprotein compounds present in such culture media may comprise one or more lipoprotein particle selected from the group consisting of (a) high density lipoprotein particles, (b) low density lipoprotein particles, and (c) very low density lipoprotein particles, as well as other types of lipoprotein particles.

Also provided herein are method for the electroporation of mammalian cell populations. Such the methods may comprising: (a) contacting the mammalian cell population with one or more lipoprotein compound for at least 12 hours (e.g., from about 12 to about 168 hours, from about 12 to about 150 hours, from about 12 to about 120 hours, from about 12 to about 100 hours, from about 12 to about 100 hours, from about 12 to about 72 hours, from about 24 to about 96 hours, from about 48 to about 150 hours, from about 48 to about 96 hours, from about 70 to about 120 hours, etc.) in a culture medium (e.g., a serum free culture medium) under conditions that allow for expansion of the mammalian cells, and (b) applying one or more electric pulse to the mammalian cell population to thereby electroporate cell membranes of members of the mammalian cell population, wherein the electroporation efficiency is at least 60% (e.g., from about 60% to about 100%, from about 60% to about 95%, from about 60% to about 90%, from about 60% to about 85%, from about 70% to about 100%, from about 70% to about 95%, from about 70% to about 90%, from about 80% to about 100%, from about 80% to about 95%, etc.) and wherein the viability of the cells in the mammalian cell population decreases by less than 10% (e.g., from about 0% to about 10%, from about 0% to about 8%, from about 0% to about 7%, from about 0% to about 5%, from about 3% to about 10%, from about 3% to about 8%, from about 3% to about 6%, from about 5% to about 10%, etc.).

In some instances, electroporation efficiency may be measured by expression of a marker (e.g., a detectable marker) in members of the mammalian cell population. Further, the marker (e.g., a detectable marker) may be a fluorescent protein (e.g., a green fluorescent protein (e.g., GFP, GFP-2, tagGFP, turboGFP, EGFP(S65T/F64L), Emerald, Azami Green, AcGFP, ZsGreen, etc.), a yellow fluorescent proteins (e.g., YFP, EYFP, mCitrine, Venus, YPet, PhiYFP, etc.), a blue fluorescent proteins (e.g., EBFP, EBFP2, Azurite, mTagBFP, etc.), a cyan fluorescent proteins (e.g., ECFP, Cerulean, CyPet, AmCyanl, Midoriishi-Cyan, etc.), a red fluorescent proteins (e.g., mPlum, AsRed2, mCherry, mRFP1, HcRed1, mRasberry, mStrawberry, Jred, etc.), an orange fluorescent proteins (e.g., mOrange, mKO2, Kusabira-Orange, mTangerine, tdTomato, etc.), or other suitable fluorescent protein.

Further provided herein are methods for the maintenance of an activated T cell population, In some instances, such methods comprise: (a) generating an activated population of T cells, (b) expanding the activated population of T cells generated in step (a) in the presence of a lipoprotein supplement, (c) exposing the expanded activated population of T cells produced in step (b) to an electric field of sufficient strength to result in a decrease in the rate of cell expansion over the following seven day by at least 30% (e.g., from about 30% to about 100%, from about 30% to about 95%, from about 30% to about 90%, from about 30% to about 85%, from about 30% to about 80%, from about 50% to about 100%, from about 50% to about 95%, from about 50% to about 85%, from about 65% to about 100%, from about 65% to about 95%, from about 60% to about 90%, from about 70% to about 100%, from about 70% to about 95%, from about 80% to about 98%, etc.), and (d) maintaining the activated population of T cells of step (c) under the same conditions as in step (b) for at least five days (e.g., seven days, from about five days to about twelve days, from about six days to about twelve days, from about six days to about ten days, from about six days to about eight days, etc.), wherein the viability of the activated population of T cells during steps (a)-(d) remains above 70% (e.g., from about 70% to about 100%, from about 80% to about 100%, from about 90% to about 100%, from about 95% to about 100%, from about 70% to about 98%, from about 80% to about 98%, from about 80% to about 95%, from about 85% to about 100%, etc.).

In some instances, one or more nucleic acid molecule (e.g., one or more nucleic acid molecule encoding a chimeric antigen receptor) may be introduced in step (c) into individual T cells of the activated population of T cells. In instances where the one or more nucleic acid molecule encodes a protein (e.g., a chimeric antigen receptor), the protein may be stably or transiently expressed within the T cells into which they are introduced.

When methods for the maintenance of an activated T cell population, for example, as set out above are practiced, then the activated population of T cells may be expanded for from about one day to about six days (e.g., from about one day to about six days, from about two days to about six days, from about three days to about six days, from about one day to about five days, from about one day to about four days, etc.) in step (b) above.

Further, methods for the maintenance of an activated T cell population, for example, as set above may further comprise: (e) washing of the activated population of T cells after step (d), and (f) expanding the washed, activated population of T cells generated in step (e) in the absence of a lipoprotein supplement.

Additionally, in many instances, the viability of the washed, activated population of T cells remains above 70% (e.g., from about 70% to about 100%, from about 80% to about 100%, from about 90% to about 100%, from about 95% to about 100%, from about 70% to about 98%, from about 80% to about 98%, from about 80% to about 95%, from about 85% to about 100%, etc.) over a five day time period, and the washed, activated population of T cells expand at least three fold (e.g., from about three fold to about twelve fold, from about four fold to about twelve fold, from about five fold to about twelve fold, from about six fold to about twelve fold, from about three fold to about ten fold, from about five fold to about eleven fold, etc.).

Methods such as those set out above allow for the storage and/or shipment of cells, while maintaining a high level of cell viability. Thus, methods are also provided herein where activated populations of T cells are shipped to a different location during step (d) (e.g., a location from about 10 to about 5,000 miles, a location from about 10 to about 100 miles, a location from about 50 to about 5,000 miles, a location from about 50 to about 3,500 miles, a location from about 200 to about 3,500 miles, a location from about 300 to about 3,500 miles, a location from about 500 to about 3,500 miles, a location from about 1,000 to about 5,000 miles, etc.).

Also, provided are methods for storing mammalian cells (e.g., T cells). Such methods may comprise the following steps (e.g., the following steps in order): (a) expanding the mammalian cells in a culture medium comprising one or more lipoprotein compound, (b) exposing the mammalian cells to an electric field, and (c) expanding the mammalian cells in a culture medium comprising one or more lipoprotein compound, wherein the mammalian cells in step (c) expand at a rate that is at least 50% (e.g., from about 50% to about 100%, from about 60% to about 100%, from about 70% to about 100%, from about 80% to about 100%, from about 50% to about 90%, from about 60% to about 90%, from about 70% to about 90%, from about 60% to about 85%, etc.) lower than in step (a), and wherein the viability of the mammalian cells remains above 70% (e.g., from about 70% to about 100%, from about 80% to about 100%, from about 90% to about 100%, from about 95% to about 100%, from about 70% to about 98%, from about 80% to about 98%, from about 80% to about 95%, from about 85% to about 100%, etc.) during steps (a)-(c).

Further, the mammalian cells may be expanded for from about one day to about six days (e.g., seven day, from about one day to about six days, from about two days to about six days, from about three days to about six days, from about one day to about five days, from about one day to about four days, etc.) days in step (c).

In some instances, the mammalian cells in step (c) may be washed and transferred to a culture medium comprising at least a 50% (e.g., from about 50% to about 100%, from about 60% to about 100%, from about 70% to about 100%, from about 80% to about 100%, from about 90% to about 100%, from about 95% to about 100%, from about 50% to about 90%, from about 60% to about 80%, etc.) lower concentration of the one or more lipoprotein compound.

In some instances, one or more nucleic acid molecule (e.g., one or more nucleic acid molecule that encodes a chimeric antigen receptor) may be introduced into the mammalian cells (e.g., T cells) in step (b).

### BRIEF DESCRIPTION OF THE DRAWINGS

FIG. 1 Human serum shows inconsistency between lots. Human T cells were expanded in basal media supplemented with several unqualified lots of human serum (labeled “huAB serum”) compared to a control lot of human serum. CTS OPTMIZER™ medium with 5% human serum. Growth was measured over a 10 day course following stimulation.

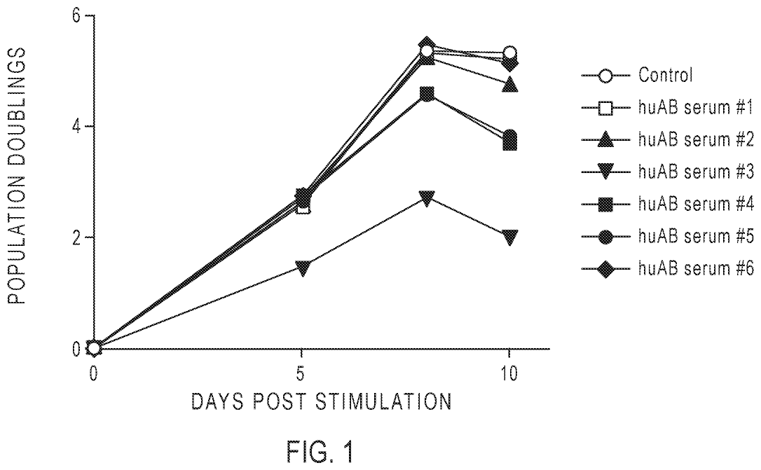

FIG. 2 shows the amino acid sequence of a human Apolipoprotein AI (SEQ ID NO: 1), as well as some regions of this protein.

FIG. 3 shows the amino acid sequence of a human Apolipoprotein AII (SEQ ID NO: 2), as well as some regions of this protein set off in outline style boxes

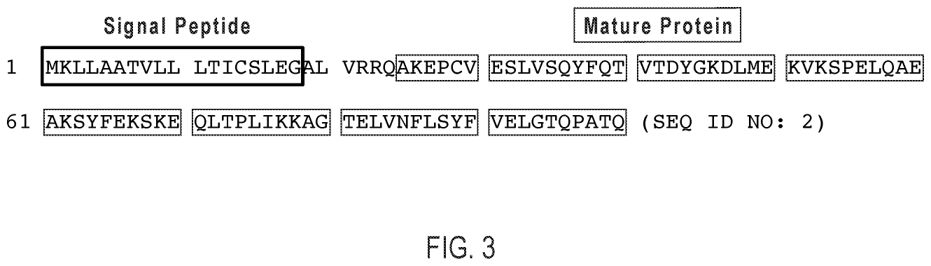

FIG. 4 shows the fold expansion at days 5 and 10 of T cells cultivated in CTS OPTMIZER™ supplemented with 8 mg/L of HDL (n=4), where the HDL is added (1) as a preformulation (“HDL in T cell Supplement”) or (2) directly to CTS OPTMIZER™ (“HDL in T cell supplement” and “HDL at point of use”, respectively) (see Example 1). Fold expansions for these two HDL additions were compared to expansion of T cells in (1) Complete CTS OPTMIZER™ and (2) X-VIVO™ 15 with 5% human serum.

FIG. 5 shows the percent viability at days 5 and 10 of T cells cultivated in basal media supplemented with 8 mg/L of HDL (n=4). Labels are as in FIG. 4.

FIG. 6 shows the CD8+/CD4+ ratio of T cells expansion for days 10 in the presence of (1) CTS OPTMIZER™ and HDL and (2) Complete CTS OPTMIZER™ (n=3). A 1.30 fold change in CD8+:CD4+ ratio was found for CTS OPTMIZER™ containing HDL, as compared to CTS OPTMIZER™.

FIG. 7 shows the phenotypes of cells expansion for days 10 in the presence of (1) CTS OPTMIZER™ and HDL and (2) Complete CTS OPTMIZER™ (n=4). A 12% increase in CD27+ T cells was found for CTS OPTMIZER™ containing HDL, as compared to Complete CTS OPTMIZER™. A 19% increase in CCR7+ T cells was found for CTS OPTMIZER™ containing HDL, as compared to Complete CTS OPTMIZER™.

FIG. 8 shows difference in viability of T cells from five different donors (D032, D093, D168, D242, and D938) that had been expanded in CTS OPTMIZER™ without ICSR containing 6 mg/L HDL (CTS OPTMIZER™ 6HDL) and CTS OPTMIZER™ complete prior to electroporation. T cell viability in CTS OPTMIZER™ complete is zero (0) on the Y axis. The T cells of all five donor samples were electroporated on day 3 (see black up arrow).

FIG. 9 shows the average total cell viability of T cells from five different donors cultured in CTS OPTMIZER™ 6HDL and CTS OPTMIZER™ complete used data presented in FIG. 8. As in FIG. 8, cells were electroporated on day 3 (see black down arrow).

FIG. 10 shows the expansion of T cells over a 10 day period in CTS OPTMIZER™ 6HDL and CTS OPTMIZER™ complete. T cells from the five donors were electroporated on day 3.

FIG. 11 shows electroporation efficiency 24 hours after electroporation of T cells from five different donors that had been expanded in CTS OPTMIZER™ 6HDL and CTS OPTMIZER™ complete.

FIG. 12 graphically shows averages of data set out in FIG. 11.

FIG. 13 shows electroporation efficiency of T cells from two different donors that were expanded prior to electroporation under various conditions. These expansion conditions are as follows: (1) CTS OPTMIZER™ without ICSR and with 6 mg/L HDL, (2) CTS OPTMIZER™ without ICSR and with 5 mg/L HDL and 1 mg/L LDL (3) CTS OPTMIZER™ without ICSR and with 4 mg/L HDL and 2 mg/L LDL, (4) CTS OPTMIZER™ without ICSR and with 3 mg/L HDL and 3 mg/L LDL, (5) CTS OPTMIZER™ without ICSR and with 2 mg/L HDL and 4 mg/L LDL, (6) CTS OPTMIZER™ without ICSR and with 1 mg/L HDL and 5 mg/L LDL, (7) CTS OPTMIZER™ without ICSR and 6 mg/L LDL, and (8) CTS OPTMIZER™ without ICSR, and (9) CTS OPTMIZER™ complete. The open down arrows show the common highest electroporation efficiencies found for the two donors.

FIG. 14 shows T cell viability under various conditions. T cells from a single donor (D032) were electroporated on day 3. The T cell sample labeled “ALL” were maintained throughout the 10 day expansion period in the same culture medium that they were contacted with pre-electroporation. Cells were washed and electroporated in OPTI-MEM™ culture medium.

FIG. 15 shows T cell viability where T cells from a single donor (D032) are cultured under various conditions before and after electroporation. T cell expansion conditions were essentially the same as in FIG. 14.

### DETAILED DESCRIPTION

### Overview

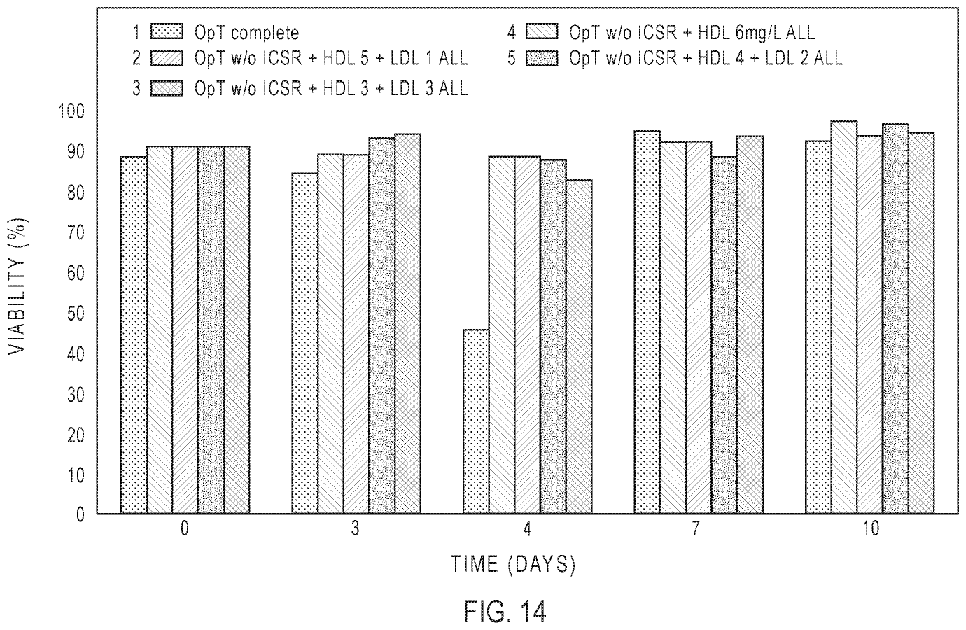

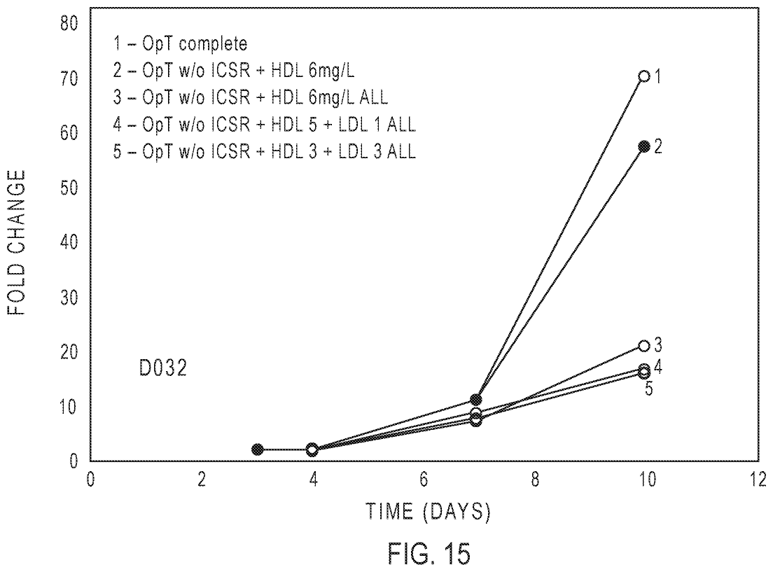

Provided herein, in part, are compositions and methods related to (1) serum-free cell culture, (2) the introduction of nucleic acid molecules into cells, and (3) the maintenance of cells at low levels of cell expansion (see FIGS. 14 and 15).

With respect to serum-free cell culture, compositions and methods are provided herein for the culture of animal cells with lipoprotein particles and/or lipoproteins. In many instances, such animal cells are cells that exhibit enhanced expansion in the presence of serum.

With respect to the introduction of nucleic acid molecules into cells, compositions and methods are provided herein for the electroporation of cells under condition that allow for increased post-electroporation cell viability and transfection efficiency. In some instances, methods set out herein involve the pre-electroporation incubation of cells with lipoprotein particles and/or lipoproteins.

### Definitions

The following definitions are included for the purpose of understanding the present subject matter and for constructing the appended patent claims. Abbreviations used herein have their conventional meaning within the chemical and biological arts.

As used herein, the term “about” in the context of a numerical value or range means±10% of the numerical value or range recited or claimed, unless the context requires a more limited range.

As used herein, the term “lipid” includes waxes, fats, oils, fatty acids, sterols, monoglycerides, diglycerides, triglycerides, phospholipids, and others. In embodiments, a lipid is a substance such as a wax, fat, oil, fatty acid, sterol, monoglyceride, diglyceride, triglyceride, or phospholipid that dissolves in alcohol but not in water. In embodiments, a lipid is a fatty acid, a glycerolipid, a glycerophospholipid, a sphingolipid, a prenol lipid, a saccharolipid, or a polyketide. In embodiments, a lipid comprises a ketoacyl or an isoprene group. In embodiments, a lipid is a wax ester. In embodiments, a lipid is an eicosanoid (e.g., a prostaglandin, a thromboxane, a leukotriene, a lipoxins, a resolvin, or an eoxin). In embodiments, a lipid is a sterol lipid. In embodiments, the sterol lipid is cholesterol or a derivative thereof. In embodiments, the cholesterol is nat-cholesterol and/or ent-cholesterol.

As used herein, the term “fatty acid” refers to a carboxylic acid (or organic acid), often with a long aliphatic tail, either saturated or unsaturated. In embodiments, a fatty acid has a carbon-carbon bonded chain of at least 4 carbon atoms in length. In embodiments, a fatty acid has a carbon-carbon bonded chain of at least 8 carbon atoms in length. In embodiments, a fatty acid has a carbon-carbon bonded chain of at least 12 carbon atoms in length. In embodiments, a fatty acid has a carbon-carbon bonded chain of at between 4 and 24 carbon atoms in length. In embodiments, a fatty acid is a naturally occurring fatty acid. In embodiments, a fatty acid is artificial (e.g., is not produced in nature). In embodiments, a naturally occurring fatty acid has an even number of carbon atoms. In embodiments, the biosynthesis of a naturally occurring fatty acid involves acetate which has two carbon atoms. In embodiments, a fatty acid may be in a free state (non-esterified) or in an esterified form such as part of a triglyceride, diacylglyceride, monoacylglyceride, acyl-CoA (thio-ester) bound or other bound form. In embodiments, the fatty acid may be esterified as a phospholipid such as a phosphatidylcholine, phosphatidylethanolamine, phosphatidylserine, phosphatidylglycerol, phosphatidylinositol or diphosphatidylglycerol form. In embodiments, a fatty acid or derivative of a fatty acid is a free fatty acid, an ester (e.g., methyl, ethyl, propyl, etc.), a mono-, di-, or triglyceride (e.g., a glycerol ester), an aldehyde, an amide, or a phospholipid version of a fatty acid disclosed herein. A “saturated fatty acid” does not contain any double bonds or other functional groups along the chain. The term “saturated” refers to hydrogen, in that all carbons (apart from the carboxylic acid [—COOH] group) contain as many hydrogens as possible. In other words, the omega end contains 3 hydrogens (CH3-) and each carbon within the chain contains 2 hydrogens (—CH2-). In an “unsaturated fatty acid,” one or more alkene functional groups exist along the chain, with each alkene substituting a singly-bonded “—CH2-CH2-” part of the chain with a doubly-bonded “—CH═CH—” portion (that is, a carbon double bonded to another carbon). The two next carbon atoms in the chain that are bound to either side of the double bond can occur in a cis or trans configuration. A table of non-limiting examples of fatty acids is as follows:

TABLE 1

| | Lipid | | Omega- |
|---|---|---|---|
| Saturation | Number | Common Name | 3, 6, or 9 |
| Saturated | 4:0 | Butyric acid | |
| | 8:0 | Caprylic acid | |
| | 10:0 | Capric acid | |
| | 12:0 | Lauric acid | |
| | 14:0 | Myristic acid | |
| | 16:0 | Palmitic acid (PA) | |
| | 18:0 | Stearic acid (SA) | |
| | 20:0 | Arachidic acid | |
| | 22:0 | Behenic acid | |
| | 24:0 | Lignoceric acid | |
| | 26:0 | Cerotic acid | |
| Monoun- | 16:1 | Palmitoleic Acid | |
| saturated | 18:1(n-9) | Oleic acid (OA) | Omega-9 |
| | 20:1(n-9) | Eicosenoic acid | Omega-9 |
| | 22:1(n-9) | Erucic acid | Omega-9 |
| | 24:1(n-9) | Nervonic acid | Omega-9 |
| Polyun- | 16:3(n-3) | Hexadecatrienoic acid (HTA) | Omega-3 |
| saturated | **18:2(n-6) | Linoleic acid (LA) | Omega-6 |
| | **18:3(n-3) | Alpha-linolenic acid (ALA) | Omega-3 |
| | **18:3(n-6) | Gamma-linolenic acid (GLA) | Omega-6 |
| | 18:4(n-3) | Stearidonic acid (SDA) | Omega-3 |
| | 20:2(n-6) | Eicosadienoic acid | Omega-6 |
| | 20:3(n-3) | Eicosatrienoic acid (ETE) | Omega-3 |
| | 20:3(n-6) | Dihomo-gamma-linolenic acid | Omega-6 |
| | | (DGLA) | |
| | 20:3(n-9) | Mead acid | Omega-9 |
| | **20:4 (n-6) | Arachidonic acid (AA) | Omega-6 |
| | 20:4(n-3) | Eicosatetraenoic acid (ETA) | Omega-3 |
| | 20:5 (n-3) | Eicosapentaenoic acid (EPA) | Omega-3 |
| | 21:5(n-3) | Heneicosapentaenoic acid (HPA) | Omega-3 |
| | 22:2(n-6) | Docosadienoic acid | Omega-6 |
| | 22:4(n-6) | Adrenic acid | Omega-6 |
| | 22:5(n-3) | Docosapentaenoic acid (DPA, | Omega-3 |
| | | Clupanodonic acid) | |
| | 22:5(n-6) | Docosapentaenoic acid | Omega-6 |
| | | (Osbond acid) | |
| | 22:6 (n-3) | Docosahexaenoic acid (DHA) | Omega-3 |
| | 24:4(n-6) | Tetracosatetraenoic acid | Omega-6 |
| | 24:5(n-3) | Tetracosapentaenoic acid | Omega-3 |
| | 24:5(n-6) | Tetracosapentaenoic acid | Omega-6 |
| | 24:6(n-3) | Tetracosahexaenoic acid | Omega-3 |
| | | (Nisinic acid) | |

As used herein, the term “lipoprotein supplement” refers to a material that contains one or more lipoprotein compound and may be added to cell culture media. Examples of lipoprotein compounds that may be present in lipoprotein supplements include lipoprotein particles, apolipoproteins and subportions thereof, synthetic HDL particle, HDL isolated from blood (e.g., human blood), and mixtures of one or more lipoprotein alone or in combination with one or more lipid and/or one or more fatty acid.

As used herein, the term “lipoprotein particles” refers to a molecular assembly that transports lipids (e.g., cholesterol and triglycerides), as well as other molecules. Lipoprotein particles with often have a phospholipid and cholesterol outer layer, with the hydrophilic portions oriented outward toward the surrounding water and lipophilic portions of each molecule oriented inwards toward the lipids molecules within the particles. Apolipoproteins are embedded in the outer layer. Thus, the complex serves to emulsify the fats. Examples of lipoprotein particles include the plasma lipoprotein particles classified as high density lipoproteins, low density lipoproteins, intermediate density lipoproteins, and very low density. Lipoprotein particles may also be generated synthetically.

As used herein, the term “high density lipoprotein” (HDL) particles refers to one of the major groups of lipoproteins. HDL particles are heterogeneous in composition and are typically composed of 80-100 proteins molecules per particle and may be composed of hundreds of lipid molecules. While there are a number of different type of naturally occurring HDL particles, these particles typically contains several types of apolipoproteins including apolipoprotein AI, apolipoprotein AII, apolipoprotein IV, apolipoprotein-CI, apolipoprotein III, apolipoprotein D, and apolipoprotein E. HDL particles are often composed of about 55% protein, from 3% to 15% triglycerides, from 26% to 46% phospholipids, from 15% to 30% cholesteryl esters and from 2% to 10% cholesterol. About 70% of the protein of HDL particles is typically apolipoprotein AI.

Based on electrophoretic migration, HDL particles can be generally classified into three subtypes. These subtypes are (1) α-migrating species (e.g., spherical HDL2 and HDL3), (2) β-migrating species (e.g., pre-β discoidal HDL, lipid-poor APO-AI, and free APO-AI), and (3) γ-migrating species.

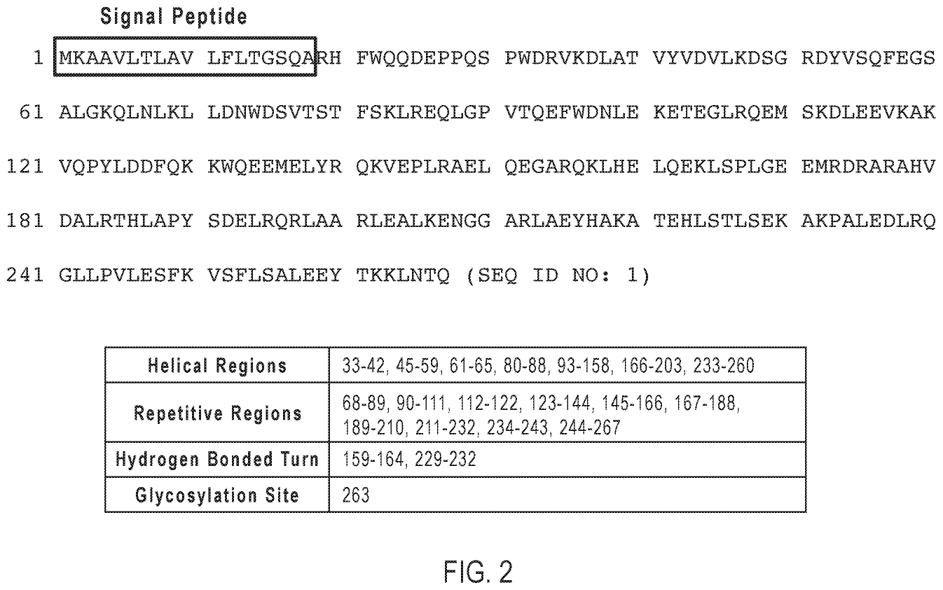

As used herein, the term “apolipoprotein AI” (APO-AI) refers to a protein that is expressed (i.e., prior to processing) in human cells with a molecular weight of about 31 kDa and consisting of 267 amino acids with aspartic acid as the N-terminal residue and glutamic acid as the C-terminal residue found in HDL particles (see, e.g., FIG. 2). There is one major APO-AI protein isoform, with a pI of 5.6, two minor isoforms, with pIs of 5.53 and 5.46), and as many as four additional isoforms. This protein has a high content of α-helix structure. Related proteins from other organisms also fall within the scope of this term.

APO-AI may be truncated at the N-terminus by from about 1 amino acid to about 30 amino acids (e.g., from about 1 amino acid to about 26 amino acids, from about 1 amino acid to about 25 amino acids, from about 1 amino acid to about 20 amino acids, from about 1 amino acid to about 19 amino acids, from about 10 amino acids to about 30 amino acids, from about 10 amino acids to about 26 amino acids, from about 10 amino acids to about 25 amino acids, from about 10 amino acids to about 19 amino acids, from about 19 amino acids to about 30 amino acids, from about 19 amino acids to about 26 amino acid, from about 18 amino acids to about 26 amino acids, etc.).

As used herein, the term “basal culture medium” or “basal culture media” refers to a cell culture medium that may be supplemented with additional components (e.g., sera, serum replacements, etc.) for improved expansion of specific cell types. Basal media may include a number of ingredients, including amino acids, vitamins, organic and inorganic salts, and sources of carbohydrate. Each ingredient may be present in an amount that supports the cultivation of cells, such amounts being generally known to a person skilled in the art. Basal media may also contain additional substances, such as buffer substances (e.g., sodium bicarbonate), antioxidants, stabilizers to counteract mechanical stress, or protease inhibitors. Exemplary basal culture media that are available from Thermo Fisher Scientifics include Advanced DMEM (cat. no. 12491-015), CTS™ KNOCKOUT™ DMEM (cat. no. A12861-01), DMEM, high glucose (cat. no. 11965-084), Advanced DMEM/F-12 (cat. no. 12634-010), CTS™ KnockOut™ DMEM/F-12 (cat. no. A13708-01), DMEM/F-12 (cat. no. 11320-033), IMEM (Improved Minimum Essential Medium) (cat. no. A10489-01), IMDM (cat. no. 12440-046), Leibovitz's L-15 Medium (cat. no. 11415-064), McCoy's 5 A (Modified) Medium (cat. no. 16600-082), MCDB 131 Medium (cat. no. 10372-019), Medium 199 (cat. no. 11150-067), Advanced MEM (cat. no. 12492-013), Fischer's Medium (cat. no. 21475-025), Advanced RPMI 1640 Medium (cat. no. 12633-012), RPMI 1640 Medium (cat. no. 11875-085), and William's E Medium (cat. no. 12551-032).

As used herein, the term “serum replacement” refers to composition that may be used in the place of serum to enhance the expansion of cells that serum enhances the expansion of. Serum replacements often contain a mixture of components. such as lipids. Examples of serum replacements include CTS™ Immune Cell SR (ICSR) (Thermo Fisher Scientific, cat. no. A2596101 and A2596102), KNOCKOUT™ Serum Replacement (Thermo Fisher Scientific, cat. no. 10828028), Serum Replacement 1 (Sigma-Aldrich, St. Louis, Mo., cat. no. S0638), and Serum Replacement Solution (PeproTech, Rocky Hill, N.J., cat. no. SR100).

Serum replacements need not be comprehensive in their components. Thus, additional components (e.g., one or more cytokine, such as Interleukin-2 (IL-2)) may be added to a basal culture medium, in addition to one or more serum replacement.

The term “immune cell” refers to a cell that may be part of the immune system and executes a particular function such as T cells, NK cells, NKT cells, B cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages. Also included are immune cells with cytotoxic function such as T cells, NK cells, NKT cells, ILC, CIK cells, LAK cells or gamma-delta T cells. Also included within the scope of “immune cells” are T cell subsets may be selected from the groups consisting of: (a) Th1 T cells, (b) Th2 T cells, (c) Th17 T cells, (d) Th22 T cells, (e) regulatory T cells, (f) naïve T cells, (g) antigen specific T cells, (h) central memory T cells, (i) effector memory T cells, (j) tissue resident memory T cells, and (k) virtual memory T cells

The term “activation,” as used herein, refers to the state of a cell following sufficient cell surface moiety ligation to induce a measurable morphological, phenotypic, and/or functional change. Within the context of T cells, such activation may be the state of a T cell that has been sufficiently stimulated to induce cellular proliferation. Activation of a T cell may also induce cytokine production and/or secretion, and up- or down-regulation of expression of cell surface molecules such as receptors or adhesion molecules, or up- or down-regulation of secretion of certain molecules, and performance of regulatory or cytolytic functions. Within the context of other cells, this term infers either up- or down-regulation of a particular physico-chemical process.

In embodiments, stimulation comprises a primary response induced by ligation of a cell surface moiety. For example, in the context of receptors, such stimulation may entail the ligation of a receptor and a subsequent signal transduction event. In embodiments, culturing T cells comprises stimulating the T cells. With respect to stimulation of a T cell, such stimulation may refer to the ligation of a T cell surface moiety that in embodiments subsequently induces a signal transduction event, such as binding the TCR/CD3 complex. In embodiments, the stimulation event may activate a cell and up- or down-regulate expression of cell surface molecules such as receptors or adhesion molecules, or up- or down-regulate secretion of a molecule, such as down-regulation of Tumor Growth Factor beta (TGF-β) or up-regulation of IL-2, IFN-γ etc. Ligands that may be used for activation include antibodies. Such antibodies may be of any species, class or subtype providing that such antibodies can react with the target of interest, e.g., CD3, the TCR, or CD28 as appropriate.

“Antibodies” for use in methods set out herein (e.g., T cell activation, immune cell purification, etc.) include:

(a) any of the various classes or sub-classes of immunoglobulin (e.g., IgG, IgA, IgM, IgD or IgE derived from any animal, e.g., any of the animals conventionally used, e.g., sheep, rabbits, goats, mice, rat, camelids, or egg yolk),

(b) monoclonal or polyclonal antibodies,

(c) intact antibodies or fragments of antibodies, monoclonal or polyclonal, the fragments being those which contain the binding region of the antibody, e.g., fragments devoid of the Fc portion (e.g., Fab, Fab′, F(ab′)2, scFv, V_{H}H, or other single domain antibodies), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains.

(d) antibodies produced or modified by recombinant DNA or other synthetic techniques, including monoclonal antibodies, fragments of antibodies, “humanized antibodies”, chimeric antibodies, or synthetically made or altered antibody-like structures.

Also included are functional derivatives or “equivalents” of antibodies e.g., single chain antibodies, CDR-grafted antibodies etc. A single chain antibody (SCA) may be defined as a genetically engineered molecule containing the variable region of the light chain, the variable region of the heavy chain, linked by a suitable polypeptide linker as a fused single chain molecule.

As used herein, the term “separation” includes any means of substantially purifying one component from another (e.g., by filtration, affinity, buoyant density, or magnetic attraction).

As used herein, the term “purifying” or “purified”, refers enhancing the amount of a component of a mixture over one or more other components. As an example, assume that Treg cells are present in a mixed population of T cells where the Treg cells represent 5% of the populations and all of the other T cells represent 95% of the total T cell population. If a process is performed that renders 20% of the population Treg cells with the other T cells representing 80% of the total T cell population, the Treg cells have been “purified”. Typically, when a T cell subset (or other cell type) has been purified, the ratio of the T cell subset (or other cell type) will be increased by at least two fold (e.g., from a 1:10 ratio to a 1:5 ratio) (e.g., from about two fold to about 100 fold, from about two fold to about 100 fold, from about 2 fold to about 100 fold, from about 5 fold to about 100 fold, from about 8 fold to about 100 fold, from about 15 fold to about 100 fold, from about 10 fold to about 40 fold, etc.).

As used herein, the term “solid support” refers to any solid phase material upon which a polypeptide, such as an antibody, may be attached for purification purposes. Thus, the term “solid support” encompasses includes resins, the wells of multiwell plates and various types of beads. In some embodiments, the configuration of the solid support is in the form of beads, spheres, particles, granules, or a surface. In some embodiments, the surface is planar, substantially planar, or non-planar. In some embodiments, solid supports may be porous or non-porous. In some embodiments, solid supports may be configured in the form of a well, depression, or other vessel. In some embodiments, solid supports may comprise a natural polysaccharide, a synthetic polymer, an inorganic material, or a combination thereof. In some embodiments, solid supports may be a bead. In some embodiments, such bead may comprise a resin that is a graft copolymer of a crosslinked polystyrene matrix and polyethylene glycol (PEG). In some embodiments, beads used in methods set out herein may be magnetic. For example, magnetization of the beads allows for one to use automated handling technologies to wash and manipulate the beads.

As used herein, “magnetic beads” refer to magnetically responsive particles that contain one or more metals or oxides or hydroxides thereof. Magnetically responsive materials of interest include paramagnetic materials, ferromagnetic materials, ferrimagnetic materials, and metamagnetic materials. In some embodiments, any magnetic beads are used, so long as these particles are dispersed or suspended in an aqueous medium and have the ability to be separated from a dispersion liquid or a suspension through application of a magnetic field. In some embodiments, magnetic beads include, for example, a salt, oxide, boride or sulfide of iron, cobalt or nickel; and rare earth elements having high magnetic susceptibility (e.g., hematite and ferrite). Specific examples of magnetic beads include iron, nickel, and cobalt.

As used, herein, the term “CD8+ T cell” refers to a T cell that presents the co-receptor CD8 on its surface. CD8 is a transmembrane glycoprotein that serves as a co-receptor for T cell receptor (TCR), which can recognize a specific antigen. Like the TCR, CD8 binds to a major histocompatibility complex I (MHC I) molecule. In embodiments, CD8+ T cells are cytotoxic CD8+ T cells (also known as cytotoxic T lymphocytes, T-killer cells, cytolytic T cells, or killer T cells). In embodiments, CD8+ T cells are regulatory CD8+ T cells, also referred to as CD8+ T cell suppressors.

As used, herein, the term “CD4+ T cell” refers to a T cell that presents the co-receptor CD4 on its surface. CD4 is a transmembrane glycoprotein that serves as a co-receptor for T cell receptor (TCR), which can recognize a specific antigen. In embodiments, CD4+ T cells are T helper cells. T helper cells (TH cells) assist other white blood cells in immunologic processes, including maturation of B cells into plasma cells and memory B cells, and activation of cytotoxic T cells and macrophages. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate different types of immune responses. Signaling from the APC directs T cells into particular subtypes. In embodiments, CD4+ T cells are regulatory T cells.

“Chimeric antigen receptor” or “CAR” or “CARs” as used herein refers to engineered receptors, which graft an antigen specificity onto cells (for example T cells such as naïve T cells, central memory T cells, effector memory T cells or any combination thereof). CARs are also known as artificial T cell receptors, chimeric T cell receptors or chimeric immunoreceptors. In embodiments, a CAR comprises one or more antigen-specific targeting domains, an extracellular domain, a transmembrane domain, one or more co-stimulatory domains, and an intracellular signaling domain. In embodiments, if the CAR targets two different antigens, the antigen-specific targeting domains may be arranged in tandem. In embodiments, if the CAR targets two different antigens, the antigen-specific targeting domains may be arranged in tandem and separated by linker sequences.

CARs are engineered receptors, which graft an arbitrary specificity onto an immune cell (e.g., a T cell, such as an activated T cell). These receptors are used to graft the specificity of a monoclonal antibody onto immune cells; with transfer of their coding sequence facilitated by retroviral vectors. The receptors are called chimeric because they are composed of parts from different sources. CARs may be used as a therapy for cancer through adoptive cell transfer. T cells are removed from a patient and modified so they express receptors specific to the patient's particular cancer. The T cells, which recognize and kill the cancer cells, are reintroduced into the patient. In embodiments, modification of T cells sourced from donors other than the patient may be used to treat the patient.

Using adoptive transfer of T cells expressing chimeric antigen receptors, CAR-modified T cells can be engineered to target any tumor-associated antigen. Following the collection of a patient's T cells, the cells are genetically engineered to express CARs specifically directed towards antigens on the patient's tumor cells before being infused back into the patient.

Some methods for engineering CAR-T cells for cancer immunotherapy use viral vectors such as retrovirus, lentivirus or transposon, which integrate the transgene into the host cell genome. Alternatively, non-integrating vectors such as plasmids or mRNA may be used but these types of episomal DNA/RNA may be lost after repeated cell division. Consequently, the engineered CAR-T cells may eventually lose their CAR expression. In another approach, a vector is used that is stably maintained in the T cell, without being integrated in its genome. This strategy has been found to enable long-term transgene expression without the risk of insertional mutagenesis or genotoxicity.

As used herein the term “homologous recombination” refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular for the repair of double strand breaks. The mechanism of homologous recombination is well known to the skilled person and has been described, for example by Paques and Haber (Paques F, Haber J E.; Microbiol. Mol. Biol. Rev. 63:349-404 (1999)). In the methods set out herein, homologous recombination is enabled by the presence of said first and said second flanking element being placed upstream (5′) and downstream (3′), respectively, of said donor DNA sequence each of which being homologous to a continuous DNA sequence within said target sequence.

As used herein the term “non-homologous end joining” (NEHJ) refers to cellular processes that join the two ends of double-strand breaks (DSBs) through a process largely independent of homology. Naturally occurring DSBs are generated spontaneously during DNA synthesis when the replication fork encounters a damaged template and during certain specialized cellular processes, including V(D)J recombination, class-switch recombination at the immunoglobulin heavy chain (IgH) locus and meiosis. In addition, exposure of cells to ionizing radiation (X-rays and gamma rays), UV light, topoisomerase poisons or radiomimetic drugs can produce DSBs. NHEJ (non-homologous end-joining) pathways join the two ends of a DSB through a process largely independent of homology. Depending on the specific sequences and chemical modifications generated at the DSB, NHEJ may be precise or mutagenic (Lieber M R., The mechanism of double-strand DNA break repair by the nonhomologous DNA end-joining pathway. Annu Rev Biochem 79:181-211).

As used herein the term “donor DNA” or “donor nucleic acid” refers to nucleic acid that is designed to be introduced into a locus by homologous recombination. Donor nucleic acid will often have at least one region of sequence homology to the locus. In many instances, donor nucleic acid will have two regions of sequence homology to the locus. These regions of homology may be at one of both termini or may be internal to the donor nucleic acid. In many instances, an “insert” region with nucleic acid that one desires to be introduced into a nucleic acid molecule present in a cell will be located between two regions of homology.

### Cell Culture Compositions

Any number of cells culture formulations may be used to prepare compositions set out herein and/or in methods set out herein.

Cell culture compositions are often designed to be modular in nature. One format is where a basal medium is prepared and one or more supplements are added to the basal medium for specific cell types and/or applications. Also, individual components (e.g., growth factors, cytokine, etc.) may be added to culture media formulations. Thus, in many instances, a fairly generic basal medium may be modified for a number of specific uses.

Components included in culture media, including mammalian cell culture media include amino acids, vitamins, glucose, buffers, salts, minerals, pH indicators (e.g., phenol red), fatty acids, sterols (e.g., cholesterol), proteins/peptides (e.g., serum albumin, insulin, insulin-like growth factor, interleukin-2, hormones, etc.), and fatty acid carriers such as cyclodextrin. The use of cyclodextrin in culture media is set out in PCT Publication WO 2019/055853, the disclosure of which is incorporated herein by reference.

### Basal Media

A considerable number of basal media have been developed over the years. Basal media will often contain basic materials for cell growth. These include vitamins and minerals. Also, a carbon sources, such as glucose, will often be present but also may be added be added.

Basal medium are generally been designed in each case on the basis of the cell type, the origin (animal species), and the purpose of the culturing. Thus, the composition of basal media can differ greatly depending on such factors.

One example of a basal medium is DMEM/F-12. The formulation of this medium is set out below in Table 2. Of course, this is only one example of a basal medium.

TABLE 2
DMEM/F-12 Formulation

| Inorganic Salts (g/liter) | | Vitamins (g/liter) | |
|---|---|---|---|
| CaCl₂ (anhydrous) | 0.11665 | D-Biotin | 0.00000365 |
| CuSO₄ (anhydrous) | 0.0000008 | Choline Chloride | 0.00898 |
| Fe(NO₃)₃·9H₂O | 0.00005 | Folic Acid | 0.00265 |
| FeSO₄·7H₂O | 0.000417 | myo-Inositol | 0.01261 |
| MgSO₄ | 0.08495 | Niacinamide | 0.00202 |
| (anhydrous) | | | |
| KCl | 0.3118 | D-Pantothenic | 0.00224 |
| | | Acid | |
| NaHCO₃ | 1.20000 | Pyridoxine·HCl | 0.00203 |
| NaCl | 7.00000 | Riboflavin | 0.00022 |
| Na₂HPO₄ | 0.07100 | Thiamine·HCl | 0.00217 |
| (anhydrous) | | | |
| NaH₂PO₄·H₂O | 0.06250 | Vitamin B-12 | 0.00068 |
| ZnSO₄·7H₂O | 0.000432 | | |

Amino Acids (g/liter)

| | | | |
|---|---|---|---|
| L-Alanine | 0.00445 | L-Leucine | 0.05895 |
| L-Arginine·HCl | 0.14750 | L-Lysine-HCl | 0.09135 |
| L-Asparagine·H₂O | 0.00750 | L-Methionine | 0.01724 |
| L-Aspartic Acid | 0.00665 | L-Phenylalanine | 0.03548 |
| L-Cysteine· | 0.01756 | L-Proline | 0.01725 |
| HCl·H₂O | | | |
| L-Cystine·2HCl | 0.03129 | L-Serine | 0.02625 |
| L-Glutamic Acid | 0.00735 | L-Threonine | 0.05355 |
| L-Glutamine | 0.36510 | L-Tryptophan | 0.00902 |
| Glycine | 0.01875 | L-Tyrosine· | 0.05582 |
| | | 2Na·2H₂O | |
| L-Histidine· | 0.03148 | L-Valine | 0.05285 |
| HCl·H₂O | | | |
| L-Isoleucine | 0.05437 | | |

Other components (g/liter)

| | | | |
|---|---|---|---|
| D-Glucose | 3.15100 | Putrescine·2HCl | 0.00008 |
| HEPES | 3.57480 | Pyruvic Acid·Na | 0.05500 |
| Hypoxanthine | 0.00239 | DL-Thioctic Acid | 0.000105 |
| Linoleic Acid | 0.000044 | Thymidine | 0.000365 |
| Phenol Red, | 0.00810 | | |
| Sodium Salt | | | |

### Culture Medium Supplements

As indicated elsewhere herein, additions made be made to basal media for specific purposes. These additions to basal will generally be made to achieve a specific purpose. Purposes include allowing for expansion of specific cell types, preferential expansion of a one or more specific cell types in a mixed population of cells, increased expansion rate of one or more specific cell types, enhanced cell viability of one or more cells types present in a mixed culture, etc.

Supplements will often be formulated for use with one or more culture medium to allow those culture media to meet at least one purpose. Some components that may be included in culture media supplements include (1) serum and tissue proteins and extracts (e.g., fetal bovine serum protein, bovine pituitary extract), (2) hydrolysates which may be animal derived (e.g., animal tissues, milk), microorganism derived (yeast), and/or plant-derived (soy, wheat, rice), (3) growth factors (e.g., EGF, FGF, IGF, NGF, PDGF, TGF), (4) hormones (e.g., growth hormone, insulin, hydrocortisone, triiodothyronine, estrogen, androgens, progesterone, prolactin, follicle-stimulating hormone, gastrin releasing peptide), (5) carrier proteins (e.g., albumin, transferrin, lactoferrin, etc.), (6) lipids and related molecules, such as cholesterol, steroids, fatty acids (e.g., palmitate, stearate, oleate, linoleate), ethanolamine, choline, inositol, etc., (7) metals (e.g., Fe, Zn, Cu, Cr, I, Co, Se, Mn, Mo, etc.), (8) vitamins (e.g., fat-soluble vitamins (A, D, E, K), water-soluble vitamins (e.g., B₁, B₂, B₆, B₁₂, C, folate), (9) polyamines, such as putrescine, spermidine, and spermine, (10) reducing agents, such as 2-mercaptoethanol, α-thioglycerol, reduced glutathione, (11) protective agents/detergents (e.g., carboxymethyl cellulose, polyvinyl pyrrolidone, Pluronic F-68, Tween 80, etc.), (12) adhesion factors, such as fibronectin and laminin, and (13) combinations of these components.

### Serum Replacements

As noted elsewhere herein, there is generally a desire to avoid the use of animal serum in cell culture systems. Further, cell culture media may be formulated to not require serum for cell cultivation or may be formulated in a modular manner so that a serum replacement may be added to the culture medium.

A number of serum replacements have been developed. These include GIBCO™ KNOCKOUT™ Serum Replacement (KNOCKOUT™ SR) (Thermo Fisher Scientific, cat. no. 10828010) and CTS™ Immune Cell SR.

Serum replacements may be animal origin free and/or immunoglobin free.

Further, serum replacements may be formulated for the cultivation of specific cell types (e.g., human embryonic stem cells, CD3+ T cells, one or more T cell subtypes, B cells, HeLa cells, 293 cells, HEK cells, etc.).

### Lipoprotein Supplements

As explained elsewhere herein, it has been found that beneficial results can be obtained from the addition of lipoprotein supplements to cell compositions.

Further, data presented herein demonstrates that lipoproteins and lipoprotein particles may act as serum replacements. Example of such serum replacements are formulation formulated and added to basal culture media in manner that results in the following components being present in the culture media in the indicated amounts: HDL (0.008 g/L), N-acetyl L cysteine (0.353 g/L), ethanolamine HCl (0.0108 g/L), human albumin (21.575 g/L), potassium chloride (0.0000216 g/L), sodium selenite (0.00000540 g/L), sodium phosphate, dibasic, 7H₂O (0.000233 g/L), potassium phosphate, monobasic (0.0000216 g/L), and sodium chloride (0.000863 g/L) (see Example 1). As discussed herein, HDL may be replaced in such culture media with other lipoprotein particles and/or one or more lipoprotein (e.g., APO-AI and/or APO-AII).

Lipoprotein supplements may be in any number of forms and may contain a number of different components. Examples of such components include one or more apolipoprotein (e.g., apolipoprotein A (e.g., APO-AI, APO-AII, apolipoprotein AIV, apolipoprotein AV), apolipoprotein B (e.g., apolipoprotein B48, apolipoprotein B100), apolipoprotein C (e.g., apolipoprotein CI, apolipoprotein CII, apolipoprotein CIII), apolipoprotein D, apolipoprotein E (e.g., apolipoprotein E-II, apolipoprotein E-IV), apolipoprotein F, apolipoprotein G, and/or apolipoprotein H).

Lipoprotein supplements may contain lipoprotein particles obtained from an animal (e.g., human, dog, cat, chimpanzee, African green monkey, chicken, etc.). Lipoprotein supplements may contain lipoprotein particles that are produced outside of an organism (i.e., synthetic lipoprotein particles).

Methods are known for the purification of lipoprotein particles. One method for purifying LDL particles is as follows. LDL particles may be isolated from 300 mls of human plasma as follows. Three mls of 100 mM EDTA is added to the plasma. The mixture is then centrifuged at 12° C. for 20 minutes at 41,000×G. The upper white layer is discarded and the lower layer is transferred to anew tube. The tube is then recentrifuged at 12° C. for 24 hours at 280,000×G. The lower layer is mixed, leaving the greenish-pellet intact. The lower level is then collected and the pellets is discarded. The density of the collected LDL-plasma is adjusted to 1.06 using Potassium Bromide (KBr). The solution is then centrifuged at 12° C. for 48 hours at 165,000×G. The uppermost fraction contains the purified LDL particles. The LDL particles may be kept under nitrogen, dark and at 4□ until use.

Weibe and Smith (“Six Methods for Isolating High-Density Lipoprotein Compared with Use of the Reference Method for Quantifying Cholesterol in Serum”, Clin. Chem. 31:746-750 (1985)), describe and compare a number of different methods for obtaining HDL particles from serum.

Lipoprotein particles may also be obtained from commercial sources. As examples, HDL and LDL particles from human blood may be purchased from Lee Biosolutions (cat. no. 361-10-0.1 and 360-10-0.1, respectively), ProSpec-Tany TechnoGene Ltd. (cat. no. PRO-559 and PRO-562, respectively)

A number of methods have been developed for the production of synthetic lipoprotein particles. One such method is set out in Tang et al., “Influence of route of administration and lipidation of apolipoprotein A-I peptide on pharmacokinetics and cholesterol mobilization”, J. Lipid Res., 58:124-136 (2017). In this paper, synthetic HDL particles by a thin film hydration method. Briefly, the phospholipids 1-palmitoyl-2-oleoyl-sn-glycero-3-phosphocholine (POPC) and 1,2-dipalmitoyl-sn-glycero-3-phosphocholine (DPPC) were dissolved in chloroform at 20 mg/ml. The APO-AI mimetic peptide 22A, PVLDLFRELLNELLEALKQKLK (SEQ ID NO: 3) was dissolved in methanol:water (1:1 volume ratio) at 10 mg/ml. DPPC, POPC, and 22A were mixed in a 4 ml glass vial at different weight ratios and vortexed for 5 seconds. The mixture was then dried by nitrogen gas flow and then placed in the vacuum oven overnight to remove residual solvent. The resulting lipid film was hydrated with PBS (pH 7.4) (final concentration of 22A=15 mg/ml) and vortexed. The suspension was homogenized in a bath sonicator for 5 min and then with a probe sonicator intermittently (50 W×10 S×12 cycles) to form a clear or translucent 22A-sHDL solution.

Methods have also been developed for the production of synthetic LDL (sLDL) (see, e.g., Hayavi and Halbert, “Synthetic Low-Density Lipoprotein, a Novel Biomimetic Lipid Supplement for Serum-Free Tissue Culture”, Biotechnol. Prog. 21:1262-1268 (2005)). In one such method, A 3:2:1 molar ratio of phosphatidylcholine, triolein, and cholesteryl oleate was dissolved in mixture dichloromethane and cholesterol, and the synthetic peptide having the following N terminal to C terminal sequence: Retinoic Acid-Leu-Arg-Leu-Thr-Arg-Lys-Arg-Gly-Leu-Lys-Leu-Cholesterol (SEQ ID NO: 4) or Retinoic Acid-Gly-Thr-Thr-Arg-Leu-Thr-Arg-Lys-Arg-Gly-Leu-Lys-Leu (SEQ ID NO: 5). These peptides were mixed at varying molar concentrations per mole with cholesteryl oleate. The dichloromethane was then added to an aqueous solution of sodium oleate and mixed at 4° C. using an EmusiFlex-05 microfluidizer (Avestin, Canada) at pressures up to 30,000 psi. The organic solvent component of the mixture was then removed at room temperature by evaporation.

A mixed sLDL (sLDL(mixed)) fatty acid system was also prepared as set out above using the following ratios of the corresponding cholesteryl ester and triglyceride, oleic (21:41)/linoleic (50:15)/palmitic (12:25)/arachidonic (6:1.3)/stearic (0:5.7), instead of pure cholesteryl oleate and triolein and Retinoic Acid-Leu-Arg-Leu-Thr-Arg-Lys-Arg-Gly-Leu-Lys-Leu-Cholesterol (SEQ ID NO: 4) at 0.03 mol/mol cholesteryl ester.

Apolipoprotein mimetic peptides that may be added to culture media compositions comprise one or more peptide set out in Table 3. Further, proteins that comprise such peptides, as well as other apolipoprotein mimetic peptides, may also be added to culture media compositions. Such proteins may be of larger size than that of peptides set out in Table 3 and may be, for example, from about 15 to about 250 (e.g., from about 15 to about 250, from about 20 to about 250, from about 30 to about 250, from about 40 to about 250, from about 60 to about 250, from about 20 to about 200, from about 20 to about 150, from about 30 to about 120, etc.) amino acids in length. Further, apolipoprotein mimetic proteins may comprise concatemers of one or more peptide set out in Table 3, as well as other apolipoprotein mimetic peptides (see Table 4).

TABLE 3
Exemplary Apolipoprotein Mimetic Peptides

| Amino Acid Sequence | SEQ ID |
|---|---|
| DWFKAFYDKVAEKFKEAF | 6 |
| EKLKAKLEELKAKLEELL | 7 |
| EKLKELLEKLLEKLKELL | 8 |
| EKLLELLKKLLELLKELL | 9 |
| EKLKELLEKLLEKLKEKL | 10 |
| EELKEKLEELKEKLEEKL | 11 |
| LRLTRKRGLKL | 12 |
| GTTRLTRKRGKL | 13 |

TABLE 4
Exemplary Apolipoprotein Mimetic
Concatemeric Peptides

| Amino Acid Sequence | SEQ ID |
|---|---|
| EKLKAKLEELKAKLEELL-EKLKAKLEELKAKLEELL | 14 |
| DWFKAFYDKVAEKFKEAF-LRLTRKRGLKL | 15 |
| LRLTRKRGLKL-EKLLELLKKLLELLKELL | 16 |

When peptides and proteins are used in culture media, these molecules may be produced by methods such as chemical synthesis or recombinantly. This will be especially desirable when animal origin free cell culture desired.

The production of recombinant proteins is well known in the art. Further, recombinant proteins may be in cells that are not of animal origin.

In some embodiments, the host cell is a non-animal, such as a plant cell. Examples of plant cells that grow readily in culture include Arabidopsis thaliana (cress), Allium sativum (garlic) Taxus chinensis, T. cuspidata, T. baccata, T. brevifolia and T. mairei (yew), Catharanthus roseus (periwinkle), Nicotiana benthamiana (solanaceae), N tabacum (tobacco) including tobacco cells lines such as NT-1 or BY-2 (NT-1 cells are available from ATCC, No. 74840, see also U.S. Pat. No. 6,140,075), Oryza sativa (rice), Cucumis sativus (cucumber), Stevia rebaudiana (sweetleaf), Stizolobium hassjoo (purselane), Panicum virgatum (switchgrass), and Zea mays spp. (maize/corn). Examples of additional host cells that may be used for recombinant protein production include organism in the following genera: Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis, Rhodoturula mucilaginosa, Phaffia rhodozyma, Xanthophyllomyces dendrorhous, Fusarium fujikuroi/Gibberella fujikuroi, Candida utilis, Candida glabrata, Candida albicans, and Yarrowia lipolytica.

### Cell Culture

Provided herein are workflows, compositions and methods for the cultivation of cells (e.g., T cells). Methods set out herein are designed for the culture of cells where the cells in culture exhibit rapid division time high levels of cell viability. In many instances, such methods may involve the culture of cells (e.g., mammalian cells) using one or more lipoprotein supplement.

As indicated elsewhere herein, cells will often be cultured in supplemented basal media. A number of components may be added to a basal medium to allow for or enhance the expansion of one or more cell type present in the medium. Such components include vitamins, minerals, lipids, growth factors, and cytokines.

There is a desire to use cell culture medium that is free of serum and free of animal origin materials. By “animal origin free” it is meant that no components are not obtained from animals or animal cells. Thus, a recombinantly expressed human protein which is produced in a yeast cell, for example, is considered to be animal origin free, even though it is a human protein. Provided herein are compositions and methods that allow for the efficient expansion of animal cells (e.g., mammalian cells) without the inclusion of serum (e.g., human serum, bovine serum, etc.). Also, provided herein are animal free compositions, and methods related thereto, that allow for the efficient expansion of animal cells (e.g., mammalian cells).

In many instances, one or more lipoprotein supplement may be added to cell culture media before, during and/or after the addition of cells. Further, one or more lipoprotein supplement may be removed from the cell culture media during the cell expansion process.

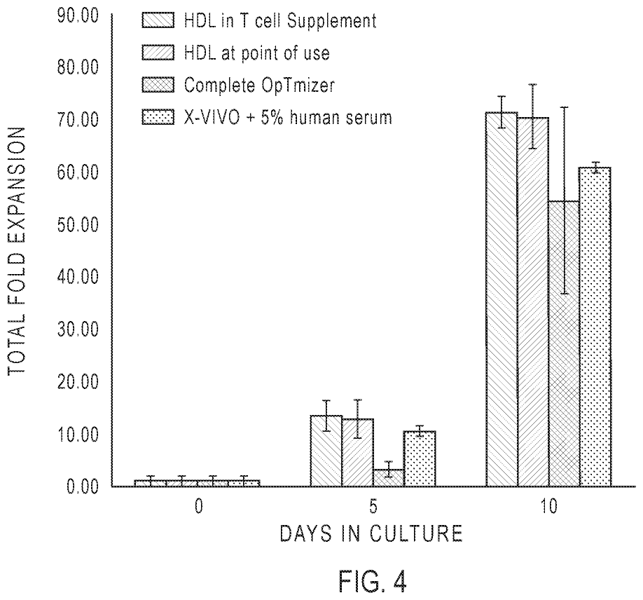

FIG. 4 shows data for the expansion of T cells in different culture media and also different culture media containing different components. The lowest level of T cell expansion was found with CTS OPTMIZER™ with ICSR (Complete CTS OPTMIZER™). The next lowest level of T cell expansion was found with X-VIVO™ with 5% human serum. The highest levels of T cell expansion were found with CTS OPTMIZER™ with 8 mg/L HDL without ICSR and result were similar for the two HDL addition data sets.

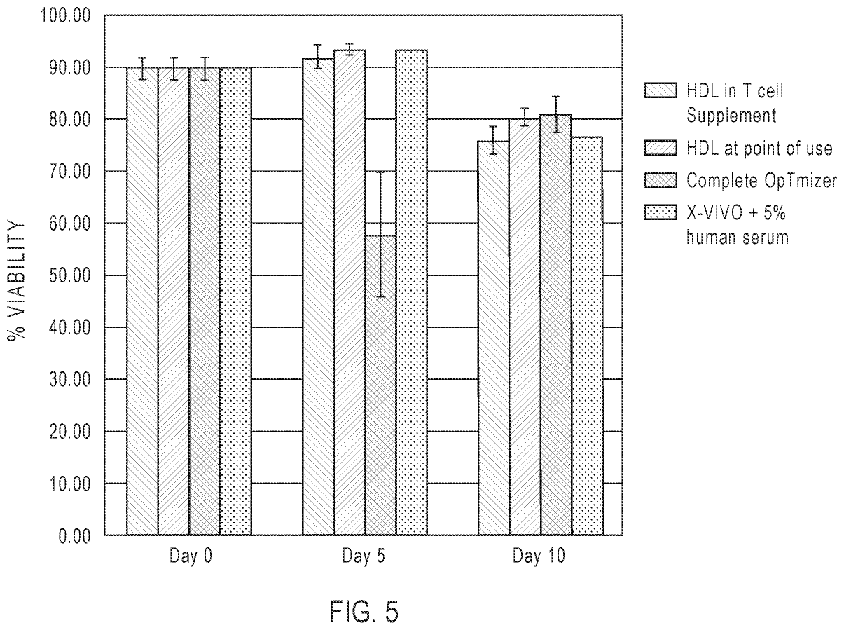

FIG. 5 shows data for the % viability of T cells related to the data set out in FIG. 4 (5). With one exception, the % of viable cells was similar in all samples and at all time points. This exception is for the day 5 measurement of the CTS OPTMIZER™ with ICSR sample.

The data set out in FIGS. 4 and 5 demonstrate that lipoprotein supplements (e.g., HDL) may be used as serum replacements. Further, lipoprotein supplements (e.g., 8 mg/L HDL) may be formulated to yield higher expansion levels in culture media than serum (e.g., human serum) or serum replacement (e.g., ICSR) and maintain cells at a higher level of viability during the expansion process.

Lipoprotein supplements added to compositions and used in methods set out herein may contain any number of components or combinations of components set our herein. In many instances, lipoprotein supplements will contain all of part of at least one lipoprotein.

Further, lipoprotein supplements may be fully of animal origin, partially of animal origin, or animal origin free. For example, lipoprotein supplements may contain one or more type of lipoprotein particle. Further, such lipoprotein particles may be derived from a naturally occurring sources (e.g., the blood of a mammal) or generated synthetically.

Lipoprotein supplements may be added to culture media to result in a final amount of component of the lipoprotein supplements in culture media. For example, lipoprotein supplements may be added to culture media to result in a final component concentration of from about 0.1 mg/L to about 500 mg/L (e.g., from about 0.2 mg/L to about 15 mg/L, from about 0.1 mg/L to about 10 mg/L, from about 0.1 mg/L to about 3 mg/L, from about 1 mg/L to about 450 mg/L, from about 1 mg/L to about 400 mg/L, from about 1 mg/L to about 350 mg/L, from about 1 mg/L to about 300 mg/L, from about 1 mg/L to about 250 mg/L, from about 1 mg/L to about 200 mg/L, from about 1 mg/L to about 150 mg/L, from about 1 mg/L to about 100 mg/L, from about 1 mg/L to about 50 mg/L, from about 1 mg/L to about 30 mg/L, from about 1 mg/L to about 20 mg/L, from about 1 mg/L to about 15 mg/L, from about 1 mg/L to about 10 mg/L, from about 3 mg/L to about 20 mg/L, from about 3 mg/L to about 15 mg/L, from about 5 mg/L to about 20 mg/L, from about 5 mg/L to about 12 mg/L, etc.).

Further, lipoprotein supplements may be added to culture media in an amount that results in specific growth characteristics. For example, lipoprotein supplements may be added in an amount that yields T cell expansion that is equal of higher than that of CTS OPTMIZER™ with ICSR (Complete CTS OPTMIZER™) at a set time point. Other growth characteristics that may be measured are % viability and the prevalence of one or more T cell subtype. Further, set time points may be three, four, five, six, seven, or ten days after the start of expansion in the presence of the lipoprotein supplement.

As an example, performance comparisons may be performed as follow. T cells from four different donors may be tested with CTS OPTMIZER™ with ICSR and CTS OPTMIZER™ with different amounts of a lipoprotein supplement (e.g., a purified apolipoprotein, HDL, LDL, etc.). At time zero activated T cells (see Example 1) are seeded at 1×10⁶ cells/well of a G-REX™ plate with 100 U/ml of IL-2. The T cells are then placed in a 37□ incubator. The T cell samples are then compared for the characteristic of interest at the set time point. For example, if the characteristic of interest is fold expansion on day five and the data set out in Table 5 obtained, then the data derived from four donors indicates that the increase in fold expansion is statistically significant and the increase in fold expansion of the CTS OPTMIZER™ with different amounts of the lipoprotein supplement sample over the CTS OPTMIZER™ with ICSR samples is 3.5. This represents an increase of 29%.

TABLE 5
(Exemplary Data): Day 5 Fold Expansion, 4 Donors (D1-D4)

| Culture Medium | Fold Expansion | Avg./SD |
|---|---|---|
| Complete CTS OPTMIZER ™ | D1 10.2, D2 12.1, | 12.0/1.40 |
| | D3 11.6, D4 14.1 | |
| CTS OPTMIZER ™ with | D1 13.2, D2 14.1, | 15.5/1.88 |
| Lipoprotein Suppl. | D3 17.6, D4 17.1 | |

In many instances, lipoprotein supplements will be added to culture media in an amount that either equals the performance of a serum replacement or exceeds the performance of a serum replacement (e.g., by from about 5% to about 100%, from about 5% to about 90%, from about 5% to about 80%, from about 5% to about 70%, from about 10% to about 100%, from about 20% to about 100%, etc.).

The lipoprotein supplement component may comprise a single protein (or peptide), a mixture of proteins, a protein fragment, a mixture of protein fragments, and/or one or more lipoprotein particle. For example, the lipoprotein supplement component may comprise a lipoprotein particle such as HDL or LDL. Further, HDL and LDL lipoprotein particles may both be added to culture media. When this is done, the concentration of either one or both of these lipoprotein particle in combination may be in the ranges indicated above or may be in the range of from about 1 mg/L to about 30 mg/L (e.g., from about 1 mg/L to about 18 mg/L, from about 1 mg/L to about 15 mg/L, from about 1 mg/L to about 10 mg/L, from about 2 mg/L to about 13 mg/L, from about 3 mg/L to about 15 mg/L, from about 5 mg/L to about 12 mg/L, etc.). Further, the ratio of two lipoprotein particles added to culture media may also vary. For example, the ratio of HDL:LDL may vary from about 10:1 to about 1:10 (e.g., from about 10:1 to about 1:10, from about 5:1 to about 1:10, from about 1:1 to about 1:10, from about 10:1 to about 1:5, from about 10:1 to about 1:1, etc.). Of course, other lipoproteins particles may also be added to culture media. Such lipoprotein particle may be obtained from natural sources (e.g., human blood) and/or may be synthetic.

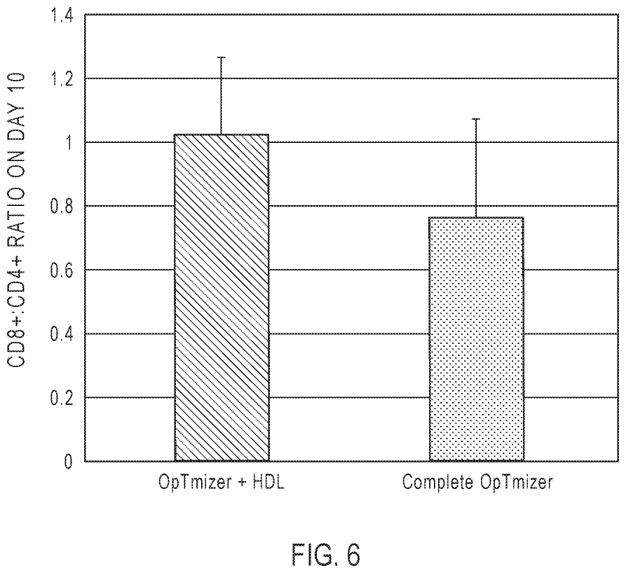

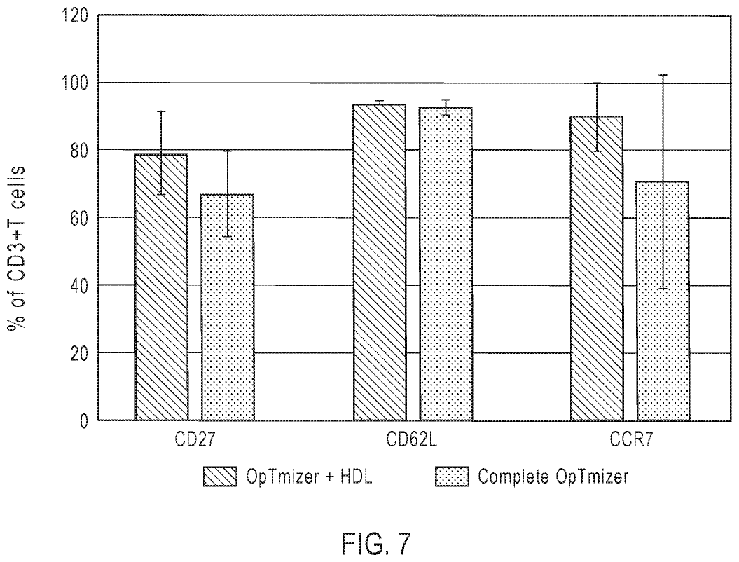

The data set out in the combination of FIG. 6 and Table 14 indicate that CD8+ T cells are preferentially expanded in CTS OPTMIZER™ with 8 mg/L HDL. The data set out in the combination of FIG. 7 and Table 15 indicate that CD27 T cells are preferentially expanded in CTS OPTMIZER™ with 8 mg/L HDL and CD62L T cells are not preferentially expanded.

Set out herein are compositions and methods for the expansion of T cells. In some instances, this expansion will result in the production of T cell populations wherein two or more T cell subtypes are present in essentially the same ratios (i.e., within about 10%) pre-expansion and post-expansion. In some instances, this expansion will result in the production of T cell populations wherein two or more T cell subtypes are present in different the same ratios (i.e., greater than about 10%, such as from about 11% to about 200%, from about 11% to about 90%, from about 11% to about 75%, from about 30% to about 200%, from about 30% to about 100%, etc.) pre-expansion and post-expansion. Further, such T cell subtypes include CD4+ T cells, CD8+ T cells, CD27+ T cells, CD62L+ T cells, and CCR7+ T cells.

As the data in Tables 16-20 indicate, individual lipoproteins may also be added to culture media as a serum replacement. The data set out in Tables 16 and 20 show that APO-AI and APO-AII function as a replacement for ICSR.

As can be seen from the data in Tables 16-20, APO-AI and APO-AII can support both T cell expansion and high levels of cell viability. These data indicate that apolipoprotein can function as serum replacements. Thus, compositions and methods are provided herein in which one or more apolipoprotein (e.g., from about one to about ten, from about two to about ten, from about three to about ten, from about one to about four, from about two to about five, etc.) and/or subportion(s) thereof are included in culture media.

### Electroporation

Provided herein are compositions and methods for the electroporation of cells. In particular, compositions and methods are provided herein which allow the electroporation of cells resulting in high post-electroporation cell viability.

A considerable amount of work has been done on mechanistic theories related to the response of cell membranes to electric field pulses that rapidly increase the transmembrane voltage, Um(t), of cell membranes to a value where cell membrane porosity dramatically rises (see Weaver et al., Bioelectrochemistry 87:236-243 (2012)). The changed in membrane porosity is believed to be caused by pore formation.

Large electric field pulses used for electroporation can kill cells either through heating or without heating being the main cause. Two non-heat killing mechanisms are believed to be via induction of apoptosis or necrosis. Further, high strength electric field cell killing is believed to be more by apoptosis, while low strength electric field cell killing is believed to be more by necrosis. Thus, it is generally desirable to adjust electrical field conditions such that high cell viability is maintained, regardless of the cell death mechanism.

Electroporation cuvettes with different “gap” sizes may be used. The “gap” is the space through which electricity is passed though. Gaps sizes may be from about 0.1 mm to about 15 mm (e.g., from about 0.5 mm to about 15 mm, from about 1 mm to about 15 mm, from about 2 mm to about 15 mm, from about 2 mm to about 10 mm, from about 2 mm to about 8 mm, from about 3 mm to about 6 mm, etc.). In many instances, a gap size of about 4 mm will be used for animal cell electroporation.

The amount of voltage applied to cells during electroporation may vary widely and maybe from about 200 Volts (V) to about 1,500 V (e.g., from about 200 V to about 1,500 V, from about 200 V to about 1,500 V, from about 250 V to about 1,500 V, from about 350 V to about 1,500 V, from about 300 V to about 1,500 V, from about 400 V to about 1,500 V, from about 500 V to about 1,500 V, from about 600 V to about 1,500 V, from about 200 V to about 1,000 V, from about 225 V to about 900 V, from about 250 V to about 900 V, from about 250 V to about 800 V, from about 300 V to about 750 V, from about 300 V to about 650 V, etc.).

Further, voltage may be applied for a variety of pulse durations. Such durations may be from about 1 nanosecond to about 1 second (e.g., from about 150 nanosecond to about 1 second, from about 250 nanosecond to about 1 second, from about 300 nanosecond to about 1 second, from about 500 nanosecond to about 800 second, from about 1 microsecond to about 1 second, from about 100 microseconds to about 1 second, from about 1 microsecond to about 800 microseconds, from about 1 microsecond to about 600 microseconds, from about 1 microsecond to about 500 microseconds, from about 1 microsecond to about 400 microseconds, from about 1 microsecond to about 300 microseconds, from about 100 microsecond to about 700 microseconds, from about 200 microsecond to about 600 microseconds, etc.).

When more than one pulse is used, the number of pulses may also vary and may be from about 1 to about 500 (e.g., from about 2 to about 500, from about 10 to about 500, from about 20 to about 500, from about 30 to about 500, from about 10 to about 250, from about 10 to about 200, from about 10 to about 170, from about 10 to about 150, from about 25 to about 250, from about 25 to about 200, from about 25 to about 150, etc.) pulses.

It has been found that the incubation of cells with lipoprotein supplements prior to electroporation can favorably modulate the effect that electroporation has on cell viability. Thus, compositions and methods are set out herein where cells are contacted with a lipoprotein supplement for a period of time, then electroporated.

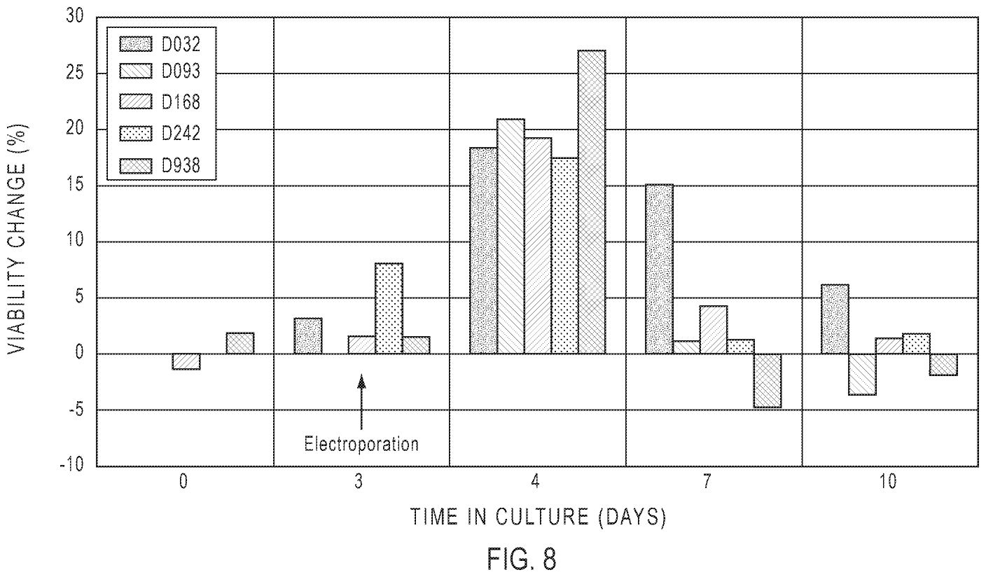

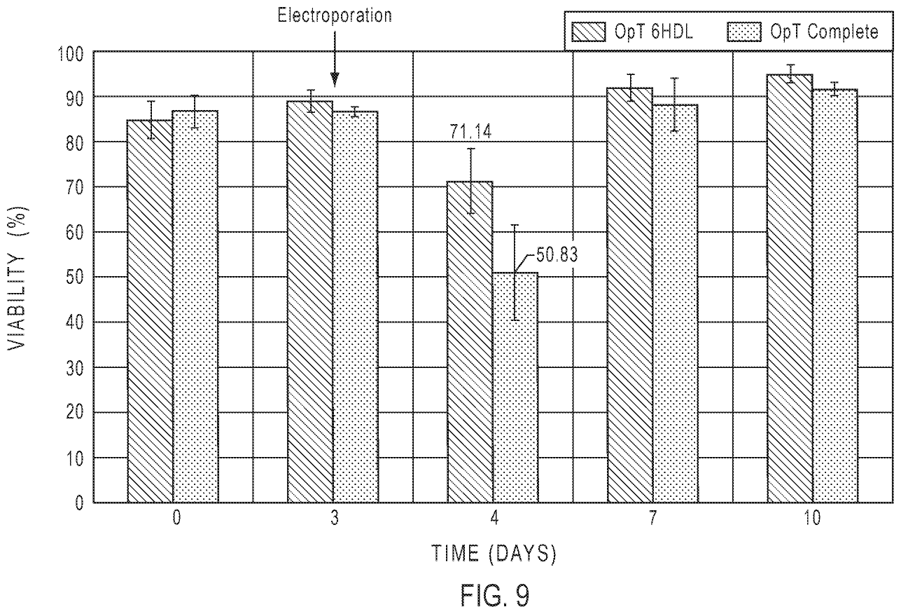

FIGS. 8 and 9 show data that were generated as set out in Example 2. T cells were expanded for three days in CTS OPTMIZER™ with 6 mg/L HDL or CTS OPTMIZER™ with ICSR. Cell viability was then measured on day 4. As can be seen, the samples in which the T cells underwent of expansion for three days with 6 mg/L HDL prior to electroporation exhibited significantly higher levels of viability than the samples in which the T cells underwent of expansion for three days in ICSR. As can be seen from FIG. 8 and Table 21, the increased T cell viability on day 4 between the two expansion conditions ranged from 20.23 and 36.21, with the average T cell viability for the T cells expanded in CTS OPTMIZER™ with 6 mg/L HDL being 70.50 and the average T cell viability for the T cells expanded in CTS OPTMIZER™ with ICSR being 49.26.

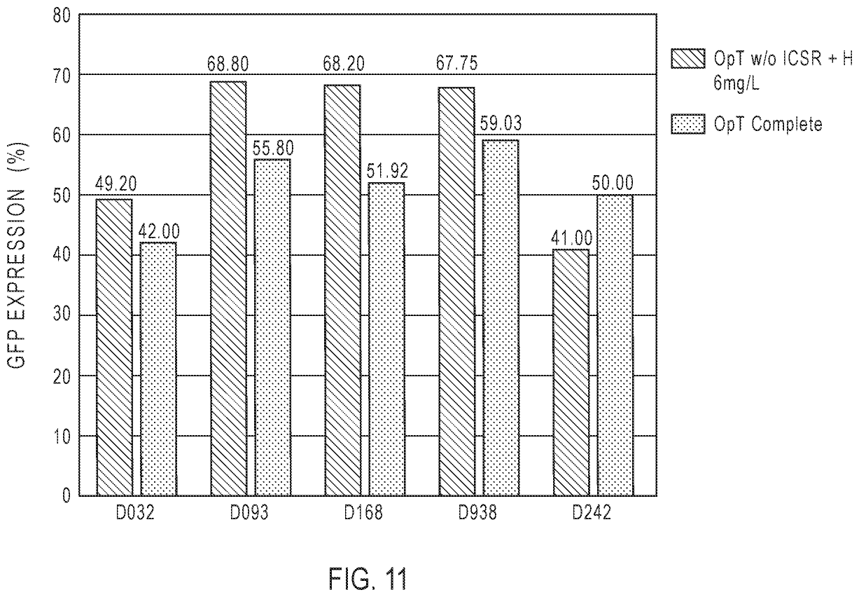

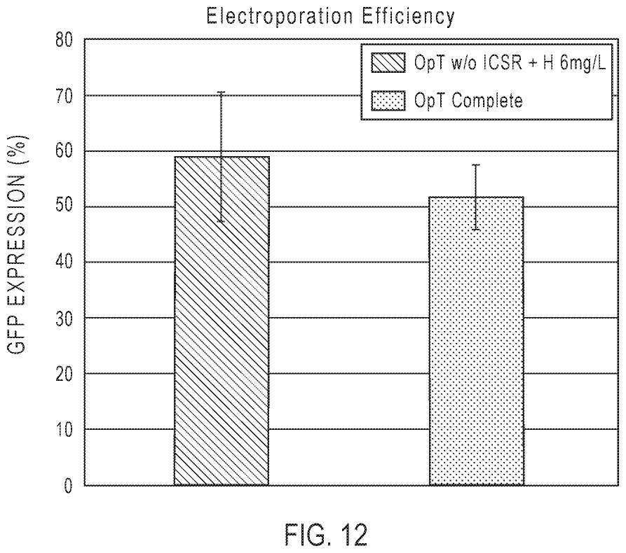

FIGS. 11 and 12 shows data for electroporation efficiency of T cells expanded for three days in CTS OPTMIZER™ with 6 mg/L HDL or CTS OPTMIZER™ with ICSR. The T cells of all but one of the donor samples exhibited increased electroporation efficiency.

Provided herein are compositions and methods for modulating the effect of electroporation on cells. In some aspects, cells are contacted with a lipoprotein supplement for a period of time (e.g., from about 1 to about 6 days, from about 1 to about 5 days, from about 1 to about 4 days, from about 1 to about 3 days, from about 2 to about 6 days, from about 2 to about 5 days, etc.) prior to electroporation. In many instances, the lipoprotein supplement will be present in a culture medium and the cells will be actively expanding during the pre-electroporation period. In some instances, the cells will be washed prior to electroporation, electroporated in a non-culture medium solution (e.g., a buffer) then resuspended in a culture medium after electroporation. In some instances, the post-electroporation culture medium will contain a lipoprotein supplement and in other instances, it will not. As an example, in some instances, T cells may be expanded in CTS OPTMIZER™ with 6 mg/L HDL for three days, washed and resuspended in a buffer, then electroporated in the buffer, then separated from the buffer and resuspended in CTS OPTMIZER™ with ICSR for further expansion. This process is essentially how the day set out in FIGS. 8-12 were generated.

The amount of lipoprotein supplement that may be added to culture media varies. In some instances, the amount will be adjusted to achieve a specified electroporation efficiency using methods set out in Example 2. Electroporation efficiency is determined by number of factors, including the cell type, the metabolic state of the cells, the nucleic acid molecule being introduced into the cells, etc.

Also, provided herein are compositions and methods for increasing the efficiency of electroporation of cells. In many instances, the amount of lipoprotein supplement that cells will be incubated with pre-electroporation are as set out elsewhere herein.

Nucleic acid molecules that may be introduced into cell by methods set out herein include RNA, DNA, and combinations thereof (RNA/DNA hybrids). Such nucleic acid molecule may be designed for transient or stable expression. Stable expression may be accomplished by the introduction of a nucleic acid molecule having, for example, an origin of replication or a nucleic acid molecule designed to integrate into the host cells genome by homologous recombination (e.g., a donor nucleic acid molecules).

Further, nucleic acid molecule introduced into cells including single-stranded DNA donor (ssDNA), blunt-end dsDNA donor (blunt), dsDNA donor with 5′ overhang (5′), and/or dsDNA donor with 3′ overhang (3′).

Nucleic acid molecule introduced into cells may encode one or more chimeric antigen receptor.

Chimeric antigen receptors (CARs) may have any number of structures and may be designed for any number of purposes. Many CARs link an extracellular antigen recognition domain to intracellular signaling domains, which activates a cell (e.g., a T cell) when an antigen is bound. CARs are often composed of three regions: An extracellular, a transmembrane domain, and an intracellular domain.

An extracellular domain is a region of CAR that is exposed to the outside of the cell and can interacts with potential target molecules. The transmembrane domain typically consisting of a hydrophobic region that spans the cell membrane (e.g., the human CD28 transmembrane domain). The intracellular domain (e.g., the cytoplasmic domain of CD3-zeta) is the internal cytoplasmic end of the receptor that “transmits” signals to the inside of the cell.

### Cell Maintenance

It has been found that cells are electroporated after incubated with lipoprotein supplements are maintained in contact with lipoprotein supplements, the cells maintain high viability for a period time but exhibit reduced expansion rates.

The data set out in FIGS. 14 and 15 was generated using T cells expanded in the indicated media. These T cells were then electroporated on Day 3. The T cells in all samples were then maintained in the indicated culture media. FIG. 14 shows data showing that when T cells are expanded in the presence of the indicated lipoprotein supplements, then electroporated and each maintained in their original culture media, cell viability remains high when lipoprotein supplements are present. FIG. 15 shows data that indicates that expansion is reduced when T cells are electroporated in the presence of lipoprotein particles (HDL and a combination of HDL and LDL), then left in contact with the lipoprotein particles. Thus, provided herein are compositions and methods for reducing the expansion rate of cells while maintaining high cell viability.

In many instances, expanding mammalian cell populations continue to expand and exhibit decreased viability conditions result in the decreased cell division. It has been observed that when cells are first expanded in the presence of a lipoprotein supplement, then placed in an electrical field, nucleic acid may be introduced into the cells with relatively low levels of loss of cell viability. Further, when such cells are maintained in culture media containing a lipoprotein supplement, these cells continue to maintain high levels of cell viability while exhibiting decreased cell expansion. Thus, compositions and methods are provided herein which allow for the expansion of mammalian cells, followed by the maintenance of cells with low levels of expansion but with high cell viability. Such compositions and methods are useful for the storage of cells.

Provided herein are methods for storing mammalian cells. Such methods included those that comprise the following steps. First, the mammalian cells are expanded in a culture medium comprising one or more lipoprotein compound for period of time (e.g., from about 1 day to about 10 days, from about 2 days to about 10 days, from about 3 days to about 10 days, from about 1 day to about 8 days, from about 1 day to about 7 days, from about 1 day to about 5 days, from about 1 day to about 4 days, from about 2 day to about 4 days, etc.). The mammalian cells are then exposing the mammalian cells to an electric field. After exposure to the electric field, the cells are maintained under conditions suitable for expanding of the mammalian cells in a culture medium comprising one or more lipoprotein compound. It has been found that the conditions for the above process may be adjusted such that the mammalian cells exhibit low levels of expansion while maintaining high levels of cell viability (see FIGS. 14 and 15).

Cell prepared for storage and stored under conditions set out herein may be any number of different cell types, including engineered cells such as T cells. These cells may be stored at 37□ during storage and may be maintained is a storage, while retaining high levels of cell viability for at least 24 days (e.g., from about 5 days to about 24 days, from about 5 days to about 20 days, from about 5 days to about 18 days, from about 5 days to about 15 days, from about 5 days to about 12 days, from about 5 days to about 10 days, from about 5 days to about 7 days, from about 1 day to about 10 days, from about 3 days to about 7 days, from about 2 days to about 8 days, etc.).

Further, at the termination of the storage period, the cells may be washed to remove the one or more lipoprotein compound and then contacted with culture media not containing a sufficient quantity of one or more lipoprotein compound to inhibit cell expansion.

Cells that may be stored by such methods include engineered T cells. T cells storage methods may be used for the transport of cells (e.g., T cells, such as engineered T cells) from one location to another.

### T Cells

Any number of different types of T cells may be present in compositions and used in methods set out herein. Some of these T cells are as follows:

Naïve T cells are generally characterized by the surface expression of L-selectin (CD62L) and C—C Chemokine receptor type 7 (CCR7); the absence of the activation markers CD25, CD44 or CD69; and the absence of memory CD45RO isoform.

Th17 Cells: T helper 17 cells (or “Th17 cells” or “Th17 helper cells”) are an inflammatory subset of CD4+ T helper cells that are believed to regulate host defense, and are involved in tissue inflammation and certain autoimmune diseases. It has been found that, when adoptively transferred into tumor-bearing mice, Th17 cells are more potent at eradicating melanoma than Th1 or non-polarized (ThO). The phenotype of Th17 cells is CD3+, CD4+, CD161+.

Memory T Cells: Memory T cells, also referred to as “antigen-experienced cells”, are experienced in a prior encounter with an antigen. These T cells are long-lived and can recognize antigens and quickly and strongly affect an immune response to an antigen to which they have been previously exposed. Memory T cells can include: Stem memory cells (TSCM), central memory cells (TCM), effector memory cells (TEM). TSCM cells have the phenotype CD45RO−, CCR7+, CD45RA+, CD62L+(L-selectin), CD27+, CD28+ and IL-7Ra+, but they also express large amounts of IL-2R, CXCR3, and LFA-1. TCM cells express L-selectin and the CCR7, they secrete IL-2, but not IFN-γ or IL-4. TEM cells do not express L-selectin or CCR7 but produce cytokines like IFN-γ and IL-4.

Memory T cell subtypes: Central memory T cells (TCM cells) express CD45RO, C—C chemokine receptor type 7 (CCR7), and L-selectin (CD62L). Central memory T cells express intermediate to high levels of CD44. This memory subpopulation is commonly found in the lymph nodes, as well as in peripheral circulation.

Tissue resident memory T cells (TRM) occupy tissues (skin, lung, gastrointestinal tract, etc.) typically without recirculating. These cells are believed to play a role in protective immunity against pathogens. Dysfunctional TRM cells have been implicated in various autoimmune diseases.

Virtual memory T cells differ from the other memory subsets in that they do not appear to originate following a strong clonal expansion event. This population as a whole is typically abundant within the peripheral circulation.

### Treatment Methods

In some aspects, methods of treating a disease in a subject in need thereof are provided herein. Such method including administering to the subject cells (e.g., T cells, NK cells, etc.) obtained or generated by methods provided herein, or progeny of such cells.

As an example, nucleic acid molecules encoding chimeric antigen receptors (CARs) may be introduced into T cells may to generate CAR-T cells. These CAR-T cells are then expanded to produce a CAR-T cell drug. T cell activation may then be mediated by the binding of antibodies the CD3 and CD28 cell surface receptors.

Any number of types of cells (e.g., natural killer (NK) cells) may be used in therapeutic methods.

NK cells are cytotoxic lymphocytes that constitute a major component of the innate immune system and are activated in response to cells signals such as interferons and macrophage-derived cytokines. The cytotoxic activity of NK cells is largely regulated by two types of surface receptors, which may be considered “activating receptors” or “inhibitory receptors,” although some receptors (e.g., CD94 and 2B4 (CD244), work either way depending on ligand interactions).

NK cells can be isolated or enriched, for example, using antibodies to CD56 and CD3, and selecting for CD56⁺CD3⁻ cells. Thus, a cell composition may be negatively selected for CD3⁻ cells, followed by positive selection for CD56⁺ cells. While both selections may be performed using solid supports to which antibodies with binding specificity to cell surface markers are bound, NK cell release need only be mediated by the positive selection step (i.e., CD56⁺ based cell purification).

As examples, NK cells play a role in the host rejection of tumors and have been shown to be capable of killing virus-infected cells. Thus, NK cells may be used in treating viral infections. Further, NK cells (e.g., activated NK cells) may be used in both ex vivo therapy and in vivo treatment of cancer.

Non-limiting examples of uses for CD8+ T cells (e.g., expanded populations of T cells comprising increased CD8+ T cell proportions, or CD8+ T cells isolated from such expanded populations) include: immunotherapies based on virus-specific T cells such as for cytomegalovirus (CMV) infection and for Epstein-Barr virus (EBV) infection for treatment of immunosuppressed transplant patients. See, e.g., Heslop et al. (2010) Blood 115(5):925-35. Additional non-limiting examples include the use of CAR-T and other modes of engineering virus-specific T cells for treatment of cancer and infectious disease. See, e.g., Pule et al. (2008) Nature Medicine 115(5):925-35 and Ghazi et al. (2013) J Immunother 35(2): 159-168. Non-limiting examples of uses for CD4+ T cells (e.g., expanded populations of T cells comprising increased CD4+ T cell proportions, or CD4+ T cells isolated from such expanded populations), include the treatment of HIV+ patients, and expanded CD4+ T helper subsets (e.g., T_{H}1, T_{H}2, T_{H}3, T_{H}17, T_{H}9, or T_{FH}), and Regulatory T cells (Treg: CD4+CD25+FoxP3+) for treating autoimmunity. See, e.g., Tebas et al. (2014) N Engl J Med 370(10):901-10 and Riley et al. (2009) Immunity 30(5): 656-665.

In some embodiments, the T cells are CD8+ T cells. In embodiments, the T cells are CD4+ T cells.

In some embodiments, T cells are isolated based upon the stage of differentiation. T cell populations may be assessed for the stage of differentiation based upon the presence or absence of certain cellular markers or proteins. Markers used to assess the stage of T cell differentiation include: CD3, CD4, CD5, CD8, CD11c, CD14, CD19, CD20, CD25, CD27, CD33, CD34, CD45, CD45RA, CD45RB, CD56, CD62L, CD123, CD127, CD278, CD335, CD11a, CD45RO, CD57, CD58, CD69, CD95, CD103, CD161, CCR7, as well as the transcription factor FOXP3.

In embodiments, once an appropriate cell population (e.g., T cell population, B cell population, etc.) or sub-population has been isolated from a patient or animal, genetic or any other appropriate modification or manipulation may optionally be carried out before the resulting cell population is expanded using compositions and methods set out herein. The manipulation may, for example, take the form of stimulate/re-stimulation of the T cells with anti-CD3 and anti-CD28 antibodies to activate/re-activate them.

In embodiments, it may be desired to administer activated cells (e.g., T cell, NK cells, etc.) to a subject and then subsequently redraw blood (or have an apheresis performed), activate and expand cells therefrom according to a method provided herein, and reinfuse the patient with these activated and expanded cells.

In embodiments, a T cell subpopulation generated according to a method provided herein may have many potential uses, including experimental and therapeutic uses. In embodiments, a small number of T cells are removed from a patient and then manipulated and expanded ex vivo before reinfusing them into the patient. Non-limiting examples of diseases that may be treated in this way are autoimmune diseases and conditions in which suppressed immune activity is desirable (e.g., for allo-transplantation tolerance). In embodiments, a therapeutic method comprises providing a mammal, obtaining a biological sample from the mammal that contains T cells; expanding/activating the T cells ex vivo in accordance with the methods provided herein; and administering the expanded/activated T cells to the mammal to be treated. In embodiments, the first mammal and the mammal to be treated can be the same or different. In embodiments, the mammal can generally be any mammal, such as a cat, dog, rabbit, horse, pig, cow, goat, sheep, monkey, or human. In embodiments, the first mammal (“donor”) can be syngeneic, allogeneic, or xenogeneic.

In embodiments, T cell subpopulations produced using the compositions and methods provided herein can be used in a variety of applications and treatment modalities. In embodiments, T cell subpopulations can be used in the treatment of disease states including, but not limited to, cancer, autoimmune disease, allergic diseases, inflammatory diseases, infectious diseases, and graft versus host disease (GVHD). In embodiments, a T cell therapy includes infusion to a subject of T cell subpopulations externally expanded by methods provided herein following or not following immune depletion, or infusion to a subject of heterologous externally expanded T cells that have been isolated from a donor subject (e.g., adoptive cell transfer).

In embodiment, where a T cell is a CAR-T cell, the selection of the antigen binding moiety may depend on the particular type of cancer to be treated. Tumor antigens are known in the art and include, for example, a glioma-associated antigen, carcinoembryonic antigen (CEA), (3-human chorionic gonadotropin, alpha fetoprotein (AFP), lectin-reactive AFP, thyroglobulin, RAGE-1, MN-CA IX, human telomerase reverse transcriptase, RUL RU2 (AS), intestinal carboxyl esterase, mut hsp70-2, M-CSF, prostate-specific antigen (PSA), PAP, NY-ESO-1, LAGE-1a, p53, prostein, PSMA, HER2/neu, surviving and telomerase, prostate-carcinoma tumor antigen-1 (PCTA-1), MAGE, ELF2M, neutrophil elastase, ephrin B2, CD22, insulin growth factor (IGF-1), IGF-II, IGF-I receptor and mesothelin.

### Examples of Sources of Mixed Population of T Cells

In embodiments, the starting source for a mixed population of T cells is blood (e.g., circulating blood) which may be isolated from a subject. In embodiments, circulating blood can be obtained from one or more units of blood or from an apheresis or leukapheresis. In embodiments, the apheresis product typically contains lymphocytes, including T cells, monocytes, granulocytes, B cells, other nucleated white blood cells, red blood cells, stem cells (e.g., induced pluripotent stem cells), and platelets. T cells, as well as other cells, can be obtained from a number of sources, including (but not limited to) blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources. T cells may also be obtained from a xenogeneic source, for example, from mouse, rat, non-human primate, and pig.

In embodiments, T cells can be obtained from a unit of blood collected from a subject using any number of techniques known to the skilled artisan, such as FICOLL™ separation. T cells may be isolated from the circulating blood of a subject. In embodiments, blood may be obtained from the subject by apheresis or leukapheresis. In embodiments, the apheresis product typically contains lymphocytes, including T cells, monocytes, granulocytes, B cells, PBMCs, other nucleated white blood cells, red blood cells, and platelets. In embodiments, prior to exposure to a sensitizing composition and subsequent activation and/or stimulation, a source of T cells is obtained from a subject. In embodiments, the cells collected by apheresis may be washed to remove the plasma fraction and to place the cells in an appropriate buffer or media for subsequent processing steps. In embodiments set out herein, cells may be washed with phosphate buffered saline (PBS). In embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the COBE® 2991 cell processor, Baxter) according to the manufacturer's instructions. In embodiments, after washing, the cells may be resuspended in a variety of biocompatible buffers, such as, for example, calcium (Ca)-free, magnesium (Mg)-free PBS. In embodiments, the undesirable components of the apheresis sample may be removed and the cells directly resuspended in culture media.

In embodiments, T cells are isolated from peripheral blood lymphocytes by lysing or removing the red blood cells and depleting the monocytes, for example, by centrifugation through a PERCOLL™ gradient. In embodiments, a specific subpopulation of T cells can be further isolated by positive or negative selection techniques.

In embodiments, T cells can be positively selected for CD3+ cells. Any selection technique known to one of skill in the art may be used. One non-limiting example is flow cytometric sorting. In another embodiment, T cells can be isolated by incubation with anti-CD3 beads. One non-limiting example is anti-CD3/anti-CD28-conjugated beads, such as CTS™ DYNABEADS® CD3/CD28 (Life Technologies Corp., Cat. No. 11141D), for a time period sufficient for positive selection of the desired T cells. In embodiments, the time period ranges from 30 minutes to 36 hours or longer and all integer values there between. In embodiments, the time period is at least 1, 2, 3, 4, 5, or 6 hours. In another embodiment the time period is 10 to 24 hours. In embodiments, the incubation time period is 24 hours. Longer incubation times, such as 24 hours, can increase cell yield. In embodiments, longer incubation times may be used to isolate T cells in any situation where there are few T cells as compared to other cell types. In embodiments, enrichment of a T cell population by negative selection can be accomplished with a combination of antibodies directed to surface markers unique to the negatively selected cells. One possible method is cell sorting and/or selection via magnetic immunoadherence or flow cytometry that uses a cocktail of monoclonal antibodies direct to cell surface markers present on the cells negatively selected. In embodiments, the fold expansion may differ based on the starting materials due to the variability of donor cells. In embodiments, the normal starting density can be between about 0.5×10⁶ to about 1.5×10⁶.

In some embodiments, T cell subpopulations may be generated by selection on the basis of whether one or more marker(s) is/are present or absent. For example, Treg cells may be obtained from a mixed population based upon the selection of cells that are CD4+, CD25+, CD127neg/low and, optionally, FOXP3+. In embodiments, Treg cells may be FOXP3−. Selection, in this instance, effectively refers to “choosing” of the cells based upon one or more definable characteristic. Further, selection can be positive or negative in that it can be for cells have one or more characteristic (positive) or for cells that do not have one or more characteristic (negative).

With respect to Treg cells, for purposes of illustration, these cells may be obtained from a mixed population through the binding of these cells to a surface (e.g., magnetic beads) having attached thereto antibodies that bind to CD4 and/or CD25 and the binding of non-Treg cells to a surface (e.g., magnetic beads) having attached thereto antibodies that binding CD127. As a specific example, magnetic beads having bound thereto an antibody that binds to CD3 may be used to isolate CD3+ cells. Once released, CD3+ cells obtained may then be contacted with magnetic beads having bound thereto an antibody that binds to CD4. The resulting CD3+, CD4+ cells may then be contacted with magnetic beads having bound thereto an antibody that binds to CD25. The resulting CD3+, CD4+, CD25+ cells may then be contacted with magnetic beads having bound thereto an antibody that binds to CD127, where the cells that are collected are those that do not bind to the beads.

In embodiments, multiple characteristics may be used simultaneously to obtain a T cell subpopulation (e.g., Treg cells). For example, a surface containing bound thereto antibodies that bind to two or more cell surface marker(s) may also be used. As a specific example, CD4+, CD25+ cells may be obtained from a mixed population through the binding of these cells to a surface having attached thereto antibodies that bind to CD4 and CD25. The selection for multiple characteristics simultaneously may result in number of undesired cells types “co-purifying” with the desired cell type(s). This is so because, using the specific example above, cells that are CD4+, CD25− and CD4−, CD25+ may be obtained in addition to CD4+, CD25+ cells.

Included herein are methods for obtaining members of one or more T cell subpopulations, where members of the T cell subpopulations are identified by specific characteristics and separated from cells which differ with respect to these characteristics. Examples of characteristics that may be used in methods set out herein include the presence or absence of the following proteins CD3, CD4, CD5, CD8, CD11c, CD14, CD19, CD20, CD25, CD27, CD33, CD34, CD45, CD45RA, CD56, CD62L, CD123, CD127, CD278, CD335, CCR7, K562P, K562CD19, and FOXP3.

### CAR-T Cells

Also provides are compositions and methods for generating chimeric antigen receptor T cells (CAR-T cells). Chimeric antigen receptors (CARs) are engineered receptors designed to provide a designated immune cell. The receptors are called chimeric because they are composed of parts from different sources.

In many instances, CAR-T cells express recombinant receptors that combine antigen-binding and T-Cell activating functions. Typically, CARs contain three regions: An extracellular domain, a transmembrane domain, and an intracellular domain.

The extracellular domain is the region of the receptor that is exposed to the exterior of the cell and if typically contains three regions: a signal peptide, an antigen recognition region, and a spacer. The signal peptide facilitates integration of the CAR into the cell membrane. The antigen recognition region of CARs is typically single-chain variable antibody fragment (e.g., an antibody fragment with binding activity for the CD19 receptor). The transmembrane domain (e.g., CD28 transmembrane domain) is typically a hydrophobic region that spans the T cell's cell membrane and allows for passage of signals received by the extracellular domain to be transmitted into the interior of the T cell. After antigen recognition, receptors cluster and a signal is transmitted to intracellular domain.

Nucleic acid molecules encoding CARs may be structured in any number of formats and may be introduced into T cells by any number of methods. CAR coding regions will normally be operably linked to expressions control sequences, such as a promoter (e.g., a CMV promoter). Further, these nucleic acid molecules will typically be present in a nucleic acid vector (e.g., a cloning vector) containing components such as elements for regulated, translation terminator, and one or more selectable markers.

One approach to treating subjects in need thereof or patients is to use the expanded T cells and genetically modify the T cells to target antigens expressed on tumor cells through the expression of CARs. In many instances, nucleic acid molecules encoding proteins, such as a CAR, will be introduced into T cells, followed by expansion of the engineered T cells.

In treatment utilizing CARs, immune cells may be collected from patient blood or other tissue. The T cells are engineered as described below to express CARs on their surface, allowing them to recognize specific antigens (e.g., tumor antigens). These CAR-T cells can then be expanded by methods set out herein and infused into the patient. Following patient infusion, the T cells will continue to expand and express the CAR, allowing for the mounting of an immune response against cells harboring the specific antigen the CAR is engineered to recognize.

Also provided herein are cells (e.g., T cells) engineered to express a CAR wherein the CAR-T cell exhibits an antitumor property. The CAR may be designed to comprise an extracellular domain having an antigen binding domain fused to an intracellular signaling domain of the T cell antigen receptor complex zeta chain (e.g., CD3 zeta). The CAR, when expressed in a T cell is able to redirect antigen recognition based on the antigen binding specificity.

The antigen binding moiety of the CAR comprises a target-specific binding element otherwise referred to as an antigen binding moiety. The choice of moiety depends on the type and number of ligands that define the surface of a target cell. For example, the antigen binding domain may be chosen to recognize a ligand that acts as a cell surface marker on target cells associated with a particular disease state. Thus, the antigen moiety domain of CARs includes those associated with viral, bacterial and parasitic infections, autoimmune disease and cancer cells.

The expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements (e.g., enhancers) regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Methods of making CAR-T cells are known in the art (see, e.g., U.S. Pat. No. 8,906,682).

### Cell Viability

A number of methods for determining cell viability are known. Such methods may be based on detection of cells that are (1) alive or dead or (2) actively proliferating. When cell populations are studies, cell viability will generally be expressed as either a percentage or ratio. As an example, if a Trypan Blue dye based assay for distinguishing between living and non-living cells is used with a population size of 100 cells and 40 cells stain with this dye and 60 cells do not stain with this dye, then 60% of the cells are viable and the ratio of non-viable cells to viable cells is 1:1.5.

Cell viability assays can be broken down into a number of categories, including the following

Membrane Disruption Assays: These assays are based upon the inability of cells to retain cellular components and/or keep materials outside of the cells. One enzyme that may be emitted by cells with disrupted cell membranes is lactate dehydrogenase. This is a stable enzyme found in many mammalian cells which can be readily detected when cell membranes are no longer intact. Trypan blue can be used as a dye exclusion assay, where this dye is not taken up by viable cells but will be taken up by non-viable cells. Trypan blue assays are advantageous because cells can be readily counted using a light microscope. Similarly to trypan blue, propidium iodide (PI) is also a membrane impermeant dye that is normally excluded from viable cells. This dye binds to double stranded DNA by intercalation. PI is excited at 488 nm and emits at a maximum wavelength of 617 nm. Due to these spectral characteristics, PI can be used with other fluorochromes, such as those excited at 488 nm (e.g., fluorescein isothiocyanate (FITC) and phycoerythrin (PE)).

7-aminoactinomycin D (7-AAD) is a fluorescent intercalator that undergoes a spectral shift upon association with DNA. 7-AAD/DNA complexes can be excited by the 488 nm laser and has an emission maxima of 647 nm, making this nucleic acid stain useful for multicolor fluorescence microscopy and flow cytometry. 7-AAD is generally excluded from live cells.

Mitochondrial Activity and Caspase Assays: A distinctive feature of the early stages of apoptosis is the disruption of the mitochondria, including changes in membrane and redox potential. MITOTRACKER™ dyes (Thermo Fisher Scientific, cat. nos. M34150, M34151, and M34152), for example, are membrane potential-dependent probes for staining mitochondria in live cells. The fluorescence signal of MITOTRACKER™ dyes is brighter in active mitochondria than in mitochondria with depolarized membranes, providing a way to identify healthy cells in a population.

Resazurin and Formazan (MTT/XTT) can assay for various stages in the apoptosis process that foreshadow cell death. ALAMARBLUE™ Cell Viability Reagent (Thermo Fisher Scientific, cat. no. DAL1025) is a ready-to-use resazurin-based solution that functions as a cell health indicator by using the reducing power of living cells to quantitatively measure viability. Resazurin, the active ingredient of ALAMARBLUE™ reagent, is a non-toxic, cell-permeable compound that is blue in color and virtually non-fluorescent. Upon entering living cells, resazurin is reduced to resorufin, a compound that is red in color and highly fluorescent. Changes in viability can be detected using either an absorbance- or fluorescence-based plate reader.

When added to cells, ALAMARBLUE™ Cell Viability Reagent is modified by the reducing environment of viable cells and turns red in color and becomes highly fluorescent. This color change and increased fluorescence can be detected using absorbance (detected at 570 nm and 600 nm) or fluorescence (using an excitation between 530-560 nm and an emission at 590 nm). To assay for viability, this reagent may be added to cells in complete media (no wash or cell lysis steps required), which are then incubated for one to four hours, and read using either an absorbance- or fluorescence-based plate reader.

One means for the detection of apoptosis is by the detection of caspase-3/7 activity. One reagent that may be sued for such detection is CELLEVENT™ Caspase-3/7 Green Detection Reagent (Thermo Fisher Scientific, cat. no. C10423). CELLEVENT™ Caspase-3/7 Green Detection Reagent is a four-amino acid peptide (DEVD (SEQ ID NO: 17)) conjugated to a nucleic acid-binding dye with absorption/emission maxima of around 502/530 nm. The DEVD peptide sequence (SEQ ID NO: 17) is a cleavage site for caspase-3/7, and the conjugated dye is non-fluorescent until cleaved from the peptide and bound to DNA. CELLEVENT™ Caspase-3/7 Green Detection Reagent is intrinsically non-fluorescent as the DEVD peptide (SEQ ID NO: 17) inhibits the ability of the dye to bind to DNA. However, after activation of caspase-3/7 in apoptotic cells, the DEVD peptide (SEQ ID NO: 17) is cleaved, enabling the dye to bind to DNA and produce a bright, fluorogenic response. The fluorescent emission of the dye when bound to DNA is around 530 nm and can be observed using a standard FITC filter set.

Functional Assays: Assays of cellular functions tends to be specific to the types of cells being assayed. As an example, motility may be used to assess sperm cell function. Gamete survival can be used to assay fertility. Red blood cells have been assayed in terms of oxygen concentration based deformability, osmotic fragility, hemolysis, hemoglobin content, and ATP level.

Nucleic Acid Incorporation Assays: These assays are based upon the incorporation of components into nucleic acid (e.g., DNA or RNA). Examples of such assays are those based on the incorporation of [³H]-thymidine or BrdU into DNA.

The selection of a cell viability assay will often be based upon a number of factors, such as cost, speed, easy of assay, reproducibility and/or reliability of the data, and the available measurement equipment. Along these lines, measurement data may be obtained, as example, using the following instruments and/or devices: light microscopy, flow cytometry, microarrays, scintillation detectors, and spectrophotometers.

The measurement of cell proliferation is generally directly related to cell viability, at least with respect to the viable cells present in the cell population. Cell proliferation and the ability of a cell to divide, are partially a measure of cell viability. With respect to a cell population, proliferation assays measure the ability of cells in the population to divide. Put another way, non-viable cells typically do not proliferate. Thus, many of the proliferating cells in a cell population are viable cells. However, most cell populations, regardless of whether cells in these populations are dividing, contain non-viable cells.

Cell proliferation may be measure by a number of different methods. Once such method is by measuring the optical density of cells being cultured in a cell culture medium. These methods are generally based upon the ability of cells to scatter light, with higher numbers of cell scattering more light. Optical density is often measured at 600 nm using a photometer.

Cell proliferation may also be performed using fluorescent dyes. One such method involves the use of CyQUANT® Cell Proliferation Assay Kit (Thermo Fisher Scientific, cat. no. C7026). The basis for of this kit is the use of a green fluorescent dye, CyQUANT® GR dye, which exhibits strong fluorescence enhancement when bound to cellular nucleic acids. Cells are lysed by addition of a buffer containing the CyQUANT® GR dye and fluorescence is then measured directly. This assay has a linear detection range extending from 50 or fewer cells to about 250,000 cells in 200 μL volumes. Excitation is typically around 485 nm and emission detection is typically around 530 nm.

### Kits

Also provided herein are kits for the culture of cells and/or for the expansion, genetic engineering, activation, storage, and electroporation macromolecules of cells. Kits provided herein may have one or more or two or more of the following components: (1) One or more cell culture medium, (2) one or more electroporation reagent, (3) one or more high density lipoprotein, (4) one or more lipoprotein compounds (e.g., HDL, LDL, APO-AI, APO-AI, etc.), (5) one or more reagent for activating T cells (e.g., a bead comprising anti-CD3 and anti-CD28 antibodies), and (6) one or more sets of instructions (e.g., written instructions) for use of kit components.

### EXAMPLES

### Example 1: Expansion of T Cells in Culture Media Containing Lipoproteins

### Materials/Methods:

High Density Lipoprotein (HDL) (Lee Biosolutions, Inc., 10850 Metro Court, Md. Heights, Mo., cat. no. 361-12) was shipped and stored at −80° C. until use, thawed in a 37° C. water bath prior to use. Three different lots were purchased and tested.

Recombinant Apolipoprotein I (APO-AI) (Abcam, 1 Kendall Square, Suite B2304, Cambridge, Mass., cat. no. ab50239) was resuspended with CTS OPTMIZER™ to a final concentration of 1 mg/mL.

Apolipoprotein II (APO-AII): APO-AII, derived from plasma, was obtained from Lee Biosolutions, Inc., and was shipped frozen, then stored at −20□, and prepared immediately prior to use by for use thawing (see HDL preparation above).

X-VIVO™ 15 (Lonza, Walkersville, Md., cat. no. 04-418Q) is a serum free medium, with L-Glutamine, gentamicin and phenol red that was formulated for hematopoietic cells.

Unless indicated otherwise, HDL, LDL, and apolipoproteins were formulated as set out in Table 6.

TABLE 6
Lipoprotein Formulation

| COMPONENTS | Final g/L and mL/L* in Culture Medium |
|---|---|
| Sodium Selenite | 0.000005332 |
| Potassium Chloride | 0.000021327 |
| Sodium Phosphate Dibasic 7H₂O | 0.000230334 |
| Potassium Phosphate Monobasic | 0.000021327 |
| Sodium Chloride | 0.000853088 |
| N Acetyl L-Cysteine | 0.348913133 |
| Human Albumin* | 21.3272086 |
| Human HDL Cholesterol | 0.008001969 |
| Ethanolamine HCl | 0.010663604 |

Cell Culture: T Cell Isolation: De-identified, frozen apheresis bags from normal donors were obtained from StemExpress (9707 Medical Center Drive, Suite 230, Rockville, Md., cat. no. LE005F). T cells were negatively isolated from PBMCs with the DYNABEADS® UNTOUCHED™ Human T Cells kit (Thermo Fisher Scientific, Cat. No. 11344D).

T Cell Activation and Expansion: T cells (seeding density 0.125×10⁶ vc/mL, 1×10⁶ vc/well in 8 mL total media) were activated with DYNABEADS® Human T-Expander CD3/CD28 (Thermo Fisher Scientific, Cat. No. 11141D) at a ratio of 3 beads per T cell and cultured in CTS OPTMIZER™ T cell Expansion Serum-Free Medium in 24-well G-REX® plates. Cells were counted on a VI-CELL™ XR analyzer (Beckman Coulter, Indianapolis Ind.).

All experiments were done in 24 well G-REX® plates (Wilson Wolf, 33 5th Ave NW, Suite 700, St Paul, Minn., P/N 80192M) except for the APO-AI experiments which were done in 24 well static plates (Corning Life Sciences, cat. no. 3524).

The following to media were used in this example:
1. CTS OPTMIZER™+HDL: 2.6% T Cell Supplement (Thermo Fisher Scientific, cat. no. A37050-01), 2 mM glutamine, 4 mM GLUTAMAX™ (Thermo Fisher Scientific, cat. no, 35050061), 8 mg/L HDL.
2. Complete CTS OPTMIZER™: 2.5% ICSR, 2.6% T Cell Supplement, 2 mM glutamine, 4 mM GLUTAMAX™.

The HDL and APO-AII experiments were done in 24 well G-REX® plates using the following protocol:

Day 0: Bulk T cells were thawed in basal CTS OPTMIZER™ without ICSR, T cell supplement, and glutamine or GLUTAMAX™ and then seed them at 1×10⁶ cells/well in total of 8 mL in each well. The T cells were then activate with DYNABEADS® Human T-Expander CD3/CD28 at 3:1 beads:cells. IL-2 was then added to 100 U/mL.

Day 3: IL-2 was re-added to an additional 100 U/mL.

Day 5: A medium exchange was performed by removal of 4 mL of the total media slowly without disturbing the cells, then fresh 4 mL of media was added to the wells. The cells we then suspended and counted using a VI-CELL™ XR analyzer (Beckman Coulter). IL-2 was also re-added to an additional 100 U/mL.

Day 7: A medium exchange was performed by removal of 4 mL of the total media slowly without disturbing the cells, 4 mL of fresh media was then added without disturbing the cells. IL-2 was also re-added to an additional 100 U/mL.

Day 10: The cells were counted using a VI-CELL™ XR analyzer. DYNABEADS® were removed from 0.5×10⁶ cells by magnetic separation. Surface staining was performed with antibodies against CD3, CD4, CD8, CD27, CCR7, and CD62L. Flow cytometric analysis was performed on a GALLIOS™ flow cytometer and KALUZA™ software.

The APO-AI experiment was performed in a 24 well static plate using the following protocol:

Day 0: Bulk T cells were thawed in basal CTS OPTMIZER™ and seed them at 1×10⁶ cells/well in total of 8 mL in each well. The T cells are then activate with DYNABEADS® Human T-Expander CD3/CD28 at 3:1 beads:cells. IL-2 was then added to 100 U/mL

Day 3, 5 and 7: The cells were counted using a VI-CELL™ XR analyzer. The cells were fed at a concentration of 0.5×10⁶ cells/mL. IL-2 was also re-added to an additional 100 U/mL after every feed.

Day 10: The cells were counted using a VI-CELL™ XR analyzer.

Phenotype Determination: Primary human T cells were expanded for 10 days with and without HDL. DYNABEADS® were removed from 0.5×10⁶ cells by magnetic separation. Surface staining was performed with antibodies against CD3, CD4, CD8, CD27, CD62L, and CCR7. Flow cytometric analysis was performed on a GALLIOS™ flow cytometer and KALUZA™ software (Beckman Coulter, Indianapolis Ind.).

### Results:

Cell Growth and Viability: T cell expansion is expressed as total fold expansion. The data set out in Tables 7 and 8 illustrate the growth of T cells in medium containing HDL without ICSR and a medium containing ICSR. Cells were expanded under two different sets of conditions. Condition 1: 8 mg/L HDL, 2.6% T Cell Supplement (Thermo Fisher Scientific, cat. no, A37050-01), 2 mM glutamine, and 4 mM GLUTAMAX™ in CTS OPTMIZER™. Condition 2: 2.6% ICSR, T Cell Supplement, 2 mM glutamine, and 4 mM GLUTAMAX™ in CTS OPTMIZER™. Results demonstrated that T cell growth is significantly increased when T cells are expanded in CTS OPTMIZER™ without ICSR but added HDL. Table 8 shows data that indicate that viability of the T cells expanded under conditions 1 and 2 significantly increases with HDL on days 5 and 7.

Experiments with HDL were performed 8 times and results show that HDL increases growth by an average of 8 fold on day 5 and 6.6 fold on day 10. It was found that HDL increases the viability by an average of 22.5% on day 5.

FIG. 4 (Tables 10 and 11) shows data were HDL was formulated in the T cell supplement to assess if HDL has the same effect on growth as adding it at point of use. T cells from four different donors were tested. The results demonstrated that HDL formulated in the T cell supplement showed the same effects on cell growth as adding HDL at point of use. The results also showed a 4.4 fold increase in growth with conditions containing HDL compared to complete CTS OPTMIZER™ on day 5 and a 1.3 fold increase in HDL compared to X-VIVO™ (Lonza, Walkersville, Md., cat. no. BEBP02-054Q) supplemented with 5% human serum. FIG. 5 (Tables 12 and 13) presents data of the viabilities of the cells expanded in the T cell supplement containing HDL, HDL at point of use, complete CTS OPTMIZER™, and X-VIVO™ supplemented with 5% human serum. The results demonstrated a 34% increase in cell viability with HDL compared to complete CTS OPTMIZER™.

FIG. 6 (Table 14) shows the CD8:CD4 ratio in cells grown with HDL and complete CTS OPTMIZER™. Results showed that there is 1.8 fold change in CD8 to CD4 ratio in conditions containing HDL compared to complete CTS OPTMIZER™.

FIG. 7 (Table 15) shows phenotypes of the cells assessed on day 10. The results show that cells containing HDL had higher CD27+ and CCR7+ phenotype compared to complete CTS OPTMIZER™ (CO).

Tables 16, 17, and 18 shows data where native APO-AII was tested in CTS OPTMIZER™ without ICSR. The results show that there is an average of 1.3 fold increase in growth on day 5 and 1.3 fold increase in growth on day 10 in conditions containing 2 μg/mL APO-AII compared to complete CTS OPTMIZER™ (CO). The viability shown in FIGS. 8A, 9A, and 10A show an increase of an average of 14.3% with conditions containing 2 μg/mL APO-AII on day 5 and an average of 9.5% on day 10.

The data set out in Table 19 was generated when T cell expansion was determined using recombinant APO-AI culture media containing ICSR. The results show a 1.1 fold increase in growth on day 10. The data in Table 20 shows a 3.5% increase in viability on day 5 and a 5.6% increase in viability on day 10 in conditions containing 1 mg/ml APO-AI+ICSR in CTS OPTMIZER™, as compared to the use of Complete CTS OPTMIZER™ (CO).

TABLE 7
Cultured T cell Fold Expansion with 8 mg/mL HDL
without ICSR and Complete CTS OPTMIZER ™ (CO)

| | | |
|---|---|---|
| Days in Culture | CO | HDL Alone |

Donor D494

| | | |
|---|---|---|
| 0 | 0.00 | 0.00 |
| 5 | 3.92 | 10.16 |
| 7 | 11.36 | 36.88 |

Donor D773

| | | |
|---|---|---|
| 0 | 0.00 | 0.00 |
| 5 | 3.28 | 8.56 |
| 7 | 13.12 | 25.52 |

Donor D242

| | | |
|---|---|---|
| 0 | 1 | 1.00 |
| 5 | 5.76 | 9.28 |
| 10 | 81.12 | 86.32 |

Donor D168

| | | |
|---|---|---|
| 0 | 1 | 1.00 |
| 5 | 3.04 | 5.36 |
| 10 | 40.72 | 57.68 |

Table/FIGURE Abbreviations:
SD—Standard Deviation
Avg—Average
XV/HS—XVIVOTM 15 + 5% human serum
CO and OC—Complete CTS OPTMIZER ™
HDL/T Cell Supp—HDL in T cell Supplement
HDL/PU—HDL/PU at point of use

TABLE 8
Viability of cultured T cells with 8 mg/mL HDL without
ICSR and Complete CTS OPTMIZER ™ (CO)

| | | |
|---|---|---|
| Day 0 | Day 5 | Day 7 |

Donor D494

| | | | |
|---|---|---|---|
| HDL alone | 62 | 89 | 89 |
| CO | 62 | 73 | 74 |

Donor D773

| | | | |
|---|---|---|---|
| HDL alone | 70 | 91 | 88 |
| CO | 70 | 71 | 77 |

TABLE 9
Viability of cultured T cells with 8 mg/mL HDL
without ICSR and Complete CTS OPTMIZER ™
(CO) (Donor D242)

| | | |
|---|---|---|
| Days in Culture | HDL Alone | CO |

Donor D242

| | | |
|---|---|---|
| 0 | 88.8% | 88.8% |
| 5 | 94.4% | 89.3% |
| 10 | 89.0% | 88.8% |

Donor D168

| | | |
|---|---|---|
| 0 | 78.1% | 78.1% |
| 5 | 95.1% | 93.0% |
| 10 | 77.3% | 80.0% |

TABLE 10
Cultured T cell Fold Expansion with 8 mg/mL HDL formulated
in the T cell Supplement without ICSR (HDL/T Cell Supp), 8 mg/mL
HDL at point of use, Complete CTS OPTMIZER ™ (CO), and
X-VIVO ™ 15 + 5% human serum (XV-HS) (see FIG. 4)

| | HDL/T | | | |
|---|---|---|---|---|
| Days | Cell Supp | HDL/PU | CO | XV-HS |
| 0 | 1 | 1 | 1 | 1 |
| 5 | 17.84 | 18.16 | 1.68 | 12.72 |
| | 12.40 | 10.88 | 4.96 | 8.24 |
| | 12.32 | 10.24 | 2.40 | 7.76 |
| | 11.60 | 12.00 | 4.00 | 13.76 |
| 10 | 73.44 | 77.76 | 31.92 | 60.16 |
| | 72.88 | 68.16 | 69.28 | 60.00 |
| | 66.96 | 63.52 | 48.48 | 54.24 |
| | 72.32 | 72.72 | 68.40 | 68.88 |

TABLE 11
Average and Standard Deviation of the Conditions above (see FIG. 4)

| | HDL/T Cell | HDL/PU | CO | XV/HS | HDL/T Cell | HDL/PU | CO | XV/HS |
|---|---|---|---|---|---|---|---|---|
| Days | Supp (Avg) | (Avg) | (Avg) | (Avg) | Supp (SD) | (SD) | (SD) | (SD) |
| 0 | 1 | 1 | 1 | 1 | 0 | 0 | 0 | 0 |
| 5 | 13.64 | 12.82 | 3.26 | 10.62 | 2.89 | 3.63 | 1.49 | 3.06 |
| 10 | 71.40 | 70.54 | 54.52 | 60.82 | 3.00 | 6.11 | 17.87 | 6.04 |

TABLE 12
Viability of Cultured T cell Fold Expansion with 8 mg/mL HDL
formulated in the T cell Supplement without ICSR, 8 mg/mL
HDL at point of use, Complete CTS OPTMIZER ™
(CO), and X-VIVO ™ 15 + 5% human serum (XV/HS) (see FIG. 5)

| Days | HDL/T Cell Supp | HDL/PU | CO | XV/HS |
|---|---|---|---|---|
| 0 | 90.5 | 90.5 | 90.5 | 90.5 |
| | 88.3 | 88.3 | 88.3 | 88.3 |
| | 88 | 88 | 88 | 88 |
| | 92.6 | 92.6 | 92.6 | 92.6 |
| 5 | 94.5 | 93.6 | 40.6 | 95.5 |
| | 92.5 | 95 | 68.8 | 94.7 |
| | 92.8 | 92.5 | 61.3 | 90.9 |
| | 88.8 | 93.2 | 60.7 | 92.8 |
| 10 | 79 | 80 | 78 | 73 |
| | 77 | 80 | 86 | 78 |
| | 75 | 83 | 80 | 79 |
| | 73 | 79 | 80 | 77 |

TABLE 13
Average and Standard Deviation of
the Conditions above (see FIG. 5)

| | HDL/T | | | | HDL/T | | | |
|---|---|---|---|---|---|---|---|---|
| | Cell | HDL/ | | XV/ | Cell | HDL/ | | XV/ |
| | Supp | PU | CO | HS | Supp | PU | CO | HS |
| Days | (Avg) | (Avg) | (Avg) | (Avg) | (SD) | (SD) | (SD) | (SD) |
| 0 | 89.9 | 89.9 | 89.9 | 89.9 | 2.15 | 2.15 | 2.15 | 2.15 |
| 5 | 92.2 | 93.6 | 57.9 | 93.5 | 2.40 | 1.05 | 12.08 | 2.06 |
| 10 | 76 | 80.5 | 81.0 | 76.8 | 2.58 | 1.73 | 3.46 | 2.63 |

TABLE 14
CD8+ to CD4+ Ratios After 10 Days of
Culture (Three Donors) (see FIG. 6)

| | | | % | | | % Ratios | |
|---|---|---|---|---|---|---|---|
| | | | Ratios | % | % | CD8+/ | |
| | % | % | (CD8+/ | CD4+ | CD8+ | CD4+ | |
| Conditions | CD4+ | CD8+ | CD4+) | (Avg) | (Avg) | (Avg) | SD |
| Day 0 | 47 | 29 | 0.62 | 41 | 35 | | |
| | 43 | 34 | 1 | | | | |
| | 33 | 42 | 0.64 | | | | |
| HDL + | 54 | 41 | 0.79 | 47.7 | 48 | 1.02 | 0.24 |
| OpTmizer | 43 | 53 | 1.23 | | | | |
| (Day 10) | 46 | 50 | 0.96 | | | | |
| CO | 65 | 32 | 1.27 | 61.3 | 35.3 | 0.77 | 0.30 |
| (Day 10) | 49 | 47 | 1.08 | | | | |
| | 70 | 27 | 0.38 | | | | |

TABLE 15
Cells Phenotype After 10 Days of Culture (Four Donors) (see FIG. 7)

| | | | | | | | SD | SD | SD |
|---|---|---|---|---|---|---|---|---|---|
| | | | | CD27 | CD62L | CCR7 | of CD27 | of CD62L | of CCR7 |
| Conditions | CD27 | CD62L | CCR7 | (Avg) | (Avg) | (Avg) | (Avg) | (Avg) | (Avg) |
| HDL + | 76 | 93 | 98 | 78.7 | 93.2 | 89.7 | 12.2 | 1.2 | 10.2 |
| OpTmizer | 85 | 92 | 79 | | | | | | |
| (Day 10) | 63 | 93 | 99 | | | | | | |
| | 91 | 95 | 83 | | | | | | |
| CO | 58 | 89 | 97 | 66.7 | 92.5 | 70.5 | 12.5 | 2.2 | 31.3 |
| (Day 10) | 77 | 94 | 40 | | | | | | |
| | 54 | 93 | 98 | | | | | | |
| | 78 | 93 | 47 | | | | | | |

TABLE 16
Cultured T cell Fold Expansion with 2 mg/L APO-
AII without ICSR and Complete CTS OPTMIZER ™ (CO)
(Donor D494)

| | | |
|---|---|---|
| Days | CO | APO-AII alone |

Donor D494

| | | |
|---|---|---|
| 0 | 0.00 | 0.00 |
| 5 | 3.92 | 4.80 |
| 7 | 11.36 | 15.84 |

Donor D773

| | | |
|---|---|---|
| 0 | 0.00 | 0.00 |
| 5 | 3.28 | 4.48 |
| 7 | 13.12 | 16.16 |

Donor 644

| | | |
|---|---|---|
| 0 | 0.00 | 0.00 |
| 5 | 3.760 | 8.080 |
| 10 | 55.800 | 56.400 |

TABLE 17
Viability of cultured T cells with 2 mg/L APO-AII
without ICSR and Complete CTS OPTMIZER ™ (CO)

| | | |
|---|---|---|
| Day 0 | Day 5 | Day 7 |

Donor D494

| | | | |
|---|---|---|---|
| APO-AII Alone | 62 | 82 | 84 |
| CO | 62 | 73 | 74 |

Donor D773

| | | | |
|---|---|---|---|
| APO-AII alone | 70 | 87 | 86 |
| CO | 70 | 71 | 77 |

Donor D644

| | | | |
|---|---|---|---|
| APO-AII | 81 | 78 | 82 |
| CO | 81 | 60 | 87 |

TABLE 18
Viability of cultured T cells with 2 mg/L APO-AII
without ICSR and Complete CTS OPTMIZER ™
(CO) (Donor D773)

| | Day 0 | Day 5 | Day 7 |
|---|---|---|---|
| APO-AII Alone | 70 | 87 | 86 |
| CO | 70 | 71 | 77 |

TABLE 19
Cultured T cell Fold Expansion with 0.1 mg/mL APO-AI with ICSR
and complete CTS OPTMIZER ™ (Donor D449)

| | Day 0 | Day 3 | Day 5 | Day 7 | Day 10 |
|---|---|---|---|---|---|
| CO | 1 | 1.27 | 5.11 | 18.45 | 83.31 |
| APO-AI + CO | 1 | 1.38 | 5.60 | 19.29 | 92.20 |

TABLE 20
Viability of cultured T cells with 0.1 mg/mL APO-AI
with ICSR and Complete CTS
OPTMIZER ™ (CO) (Donor D449)

| | Day 0 | Day 3 | Day 5 | Day 7 | Day 10 |
|---|---|---|---|---|---|
| CO | 72.60% | 88.60% | 78.20% | 80.50% | 74.70% |
| APO-AI + CO | 72.60% | 89.70% | 81.70% | 80.70% | 80.30% |

### Example 2: Electroporation of Cells Expanded with Lipids

### Methods

Unless indicated, the following methods were used in this example. Also, in this example HDL was obtained from Lee Biosolutions, Inc., 10850 Metro Court, Maryland Heights, Mo. (cat. nos. 361-10 and 361-12) and added directly to media without further dilution.

T cells were activated using (1) beads comprising anti-CD3 and anti-CD28 antibodies beads (Thermo Fisher Scientific, cat. no. 11131D) and (2) IL-2 (100 IU/mL) (Thermo Fisher Scientific, cat. no. CTP0021) for 3 days in recovery media (CTS OPTMIZER™ without ICSR with 6 mg/L HDL, referred to herein as “CTS OPTMIZER™ 6HDL”) or CTS OPTMIZER™ complete (CTS OPTMIZER™ with ICSR) as a control. On day 3, cells were counted, washed then resuspended in OPTI-MEM™ cell culture medium (Thermo Fisher Scientific, cat. no. 11058021) and electroporated (Neon Transfection System, 1100V, two pulse lengths of 20 ms each). After electroporation, cells were incubated into CTS OPTMIZER™ complete media with IL-2 (100 IU/mL). Cell viability and electroporation efficiency (GFP expression) was determined 24 hours after electroporation using an pAAV-GFP vector (Cell Biolabs Inc., cat. no. AAV-400). GFP expression was determined using flow cytometry (Beckman Coulter, GALLIOS™ instrument). Cell were counted on day 7 and transfer to new 12 well static plates at the concentration of 0.5×10⁶/ml with fresh media and IL-2 (100 IU/mL) and cultured until day 10, when viability and counts were determined.

Data set out in FIGS. 14 and 15 were generated with the following variations. In these experiments, cells were continually contacted with the indicated culture media through out the 10 day workflow.

### Results

In some of the experiments set out herein, data variations were found between individual donors. Such variations can be seen in FIG. 8, which shows data generated using T cells from five different donors.

The data set out in FIG. 8 represents a comparison if viability data between T cells cultured in CTS OPTMIZER™ 6HDL and CTS OPTMIZER™ complete. The base line (zero) at each time point and for each donor was set by cell viability measurement of T cells in CTS OPTMIZER™ complete. Thus, the height of each bar reflects a difference in viability.

The data in FIG. 8 show that the greatest difference in viability is seen 24 hours after electroporation, with the average enhancement in T cell viability for the CTS OPTMIZER™ 6HDL samples being around 20%. As can be seen from the data, the viability of T cells expanded in CTS OPTMIZER™ 6HDL prior to electroporation in is higher for all five donor samples 24 hours after electroporation than for T cells expanded in CTS OPTMIZER™ complete. These data demonstrate that the pre-electroporation expansion of T cells with 6 mg/L HDL results in increased cell viability 24 hours after electroporation.

The data set out in FIG. 9 also demonstrate that pre-electroporation expansion of T cells with 6 mg/L HDL results in higher cell viability after electroporation. In particular, the data represented in FIG. 9 show a lower average decrease in cell viability 24 hours after electroporation for CTS OPTMIZER™ 6HDL than for CTS OPTMIZER™ complete. For CTS OPTMIZER™ 6HDL, the average cell viability at days 3 and 7 was shown to be around 90% (Day 3: 88.95%, SD 2.42; Day 7: 91.83%, SD 3.08), this decreases on day 4 to an average of around 70% (71.14%, SD 7.26). For CTS OPTMIZER™ complete, the average cell viability at days 3 and 7 was shown to be around 87% (Day 3: 86.57%, SD 1.13; Day 7: 88.17%, SD 5.79), this decreases on day 4 to an average of around 50% (50.83%, SD 10.54). Thus, the decrease in viability on day 4 for CTS OPTMIZER™ 6HDL is around 18% (17.81%) and the decrease in viability on day 4 for CTS OPTMIZER™ complete is around 36% (35.74%). Thus, expansion of cells in CTS OPTMIZER™ 6HDL resulted in about 50% higher cell viability than in CTS OPTMIZER™ complete 24 hours after electroporation.

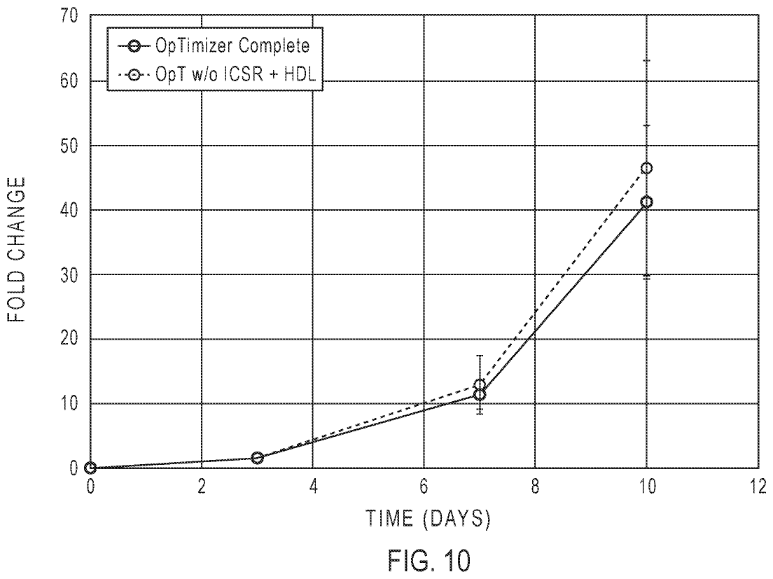

The data set out in FIG. 10 show that T cells in expanded in CTS OPTMIZER™ 6HDL prior to electroporation achieve higher expansion at day 10 (46.34 fold expansion, SD 16.62 vs. 41.05 fold expansion, SD 11.83; respectively) than T cells expanded in CTS OPTMIZER™ complete.

FIGS. 11 and 12 show comparisons of electroporation efficiency of T cells expanded prior to electroporation in CTS OPTMIZER™ 6HDL and CTS OPTMIZER™ complete. Electroporation efficiencies were found to be an average of 58.99%, SD 11.64 for CTS OPTMIZER™ 6HDL and 51.74%, SD 5.79 for CTS OPTMIZER™ complete. Thus, an increase in electroporation efficiency of about 7% was observed for CTS OPTMIZER™ 6HDL as compared to CTS OPTMIZER™ complete.

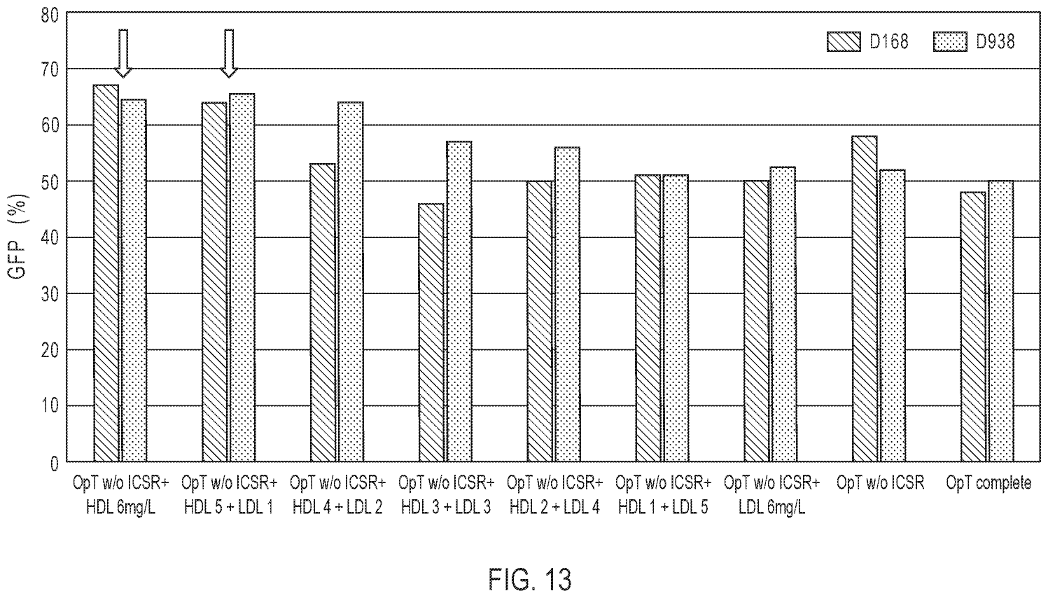

The data set out in FIG. 13 show electroporation efficiency comparisons for T cell obtained from two donors under different conditions. The highest consistent electroporation efficiencies were seen for CTS OPTMIZER™ 6HDL and CTS OPTMIZER™ without ICSR with 5 mg/L HDL and 1 mg/L LDL.

The data set out in FIG. 14 shows that T cell viability is maintained over a seven day period after electroporation when cells are kept in contact with HDL and LDL in CTS OPTMIZER™ without ICSR. The data in FIG. 15 shows that expansion of the T cells kept in contact with HDL and LDL in CTS OPTMIZER™ without ICSR was significantly slower than for T cells transferred to CTS OPTMIZER™ complete after electroporation. Thus, the data set out in FIGS. 14 and 15 demonstrate that nucleic acid molecules may be introduced into T cells and the cells may be maintained for at least seven days in a low expansion/high viability state.

TABLE 21
Data used to generate FIG. 8.

| | | Viability (%) | | | |
|---|---|---|---|---|---|
| | | OpT | OpT | | % |
| Donor | Day | 6HDL | complete | Difference | Difference |
| D032 | 0 | 80.95 | 80.95 | 0.00 | 0 |
| | 3 | 90.76 | 87.55 | 3.21 | 3.54 |
| | 4 | 66.97 | 48.60 | 18.37 | 27.43 |
| | 7 | 92.29 | 77.03 | 15.26 | 16.53 |
| | 10 | 95.60 | 89.40 | 6.20 | 6.48 |
| D093 | 0 | 91.10 | 91.10 | 0.00 | 0 |
| | 3 | 87.30 | 87.30 | 0.00 | 0 |
| | 4 | 58.00 | 37.00 | 21.00 | 36.21 |
| | 7 | 92.70 | 91.60 | 1.10 | 1.20 |
| | 10 | 90.80 | 94.40 | −3.60 | 3.96 |
| D168 | 0 | 88.25 | 89.58 | −1.33 | 1.51 |
| | 3 | 89.72 | 88.12 | 1.60 | 1.78 |
| | 4 | 59.90 | 40.57 | 19.32 | 32.25 |
| | 7 | 94.06 | 89.76 | 4.29 | 4.56 |
| | 10 | 96.04 | 94.63 | 1.42 | 1.51 |
| D242 | 0 | 83.40 | 83.40 | 0.00 | 0 |
| | 3 | 93.80 | 85.70 | 8.10 | 8.63 |
| | 4 | 86.50 | 69.00 | 17.50 | 20.23 |
| | 7 | 96.23 | 94.95 | 1.28 | 1.33 |
| | 10 | 97.50 | 95.70 | 1.80 | 1.85 |
| D938 | 0 | 89.45 | 87.60 | 1.85 | 2.07 |
| | 3 | 86.75 | 85.22 | 1.53 | 1.76 |
| | 4 | 78.21 | 51.14 | 27.08 | 34.62 |
| | 7 | 80.19 | 84.94 | −4.75 | 5.92 |
| | 10 | 90.69 | 92.54 | −1.85 | 2.04 |

TABLE 22
T Cell Viability (Data used to generate FIG. 9).

| Medium | Day | Viability | SD |
|---|---|---|---|
| OpT Complete | 0 | 86.69 | 3.49 |
| | 3 | 86.57 | 1.13 |
| | 4 | 50.83 | 10.54 |
| | 7 | 88.17 | 5.79 |
| | 10 | 91.62 | 1.40 |
| OpT 6HDL | 0 | 84.90 | 4.08 |
| | 3 | 88.95 | 2.42 |
| | 4 | 71.14 | 7.26 |
| | 7 | 91.83 | 3.08 |
| | 10 | 94.88 | 2.05 |

TABLE 23
T Cell Viability 24 Hours/Day 4 After
Electroporation (Data used to generate FIG. 9).

| | | Viability | |
|---|---|---|---|
| Medium | Donor | (%) | Stats |
| OpT complete | (D242 + D938 + D092) | 53.97 | 50.83% |
| | D032 | 48.60 | (mean) |
| | D093 | 37.00 | 10.54 |
| | D168 | 40.57 | (SD) |
| | D242 | 69.00 | |
| | D938 | 55.85 | |
| OpT 6HDL | (D242 + D938 + D092) | 67.82 | 71.14% |
| | D032 | 70.58 | (mean) |
| | D168 | 63.30 | 7.95 |
| | D242 | 86.50 | (SD) |
| | D938 | 68.64 | |
| | D093 | 70.00 | |

TABLE 24
T Cell Expansion in OpT complete and OpT
6HDL (Data used to generate FIG. 10).

| | | Fold | |
|---|---|---|---|
| Day | Media | Change | SD |
| 0 | OpT Complete | 0.00 | 0.00 |
| 3 | | 1.63 | 0.44 |
| 7 | | 11.39 | 2.28 |
| 10 | | 41.05 | 11.83 |
| 0 | OpT 6HDL | 0.00 | 0.00 |
| 3 | | 1.59 | 0.44 |
| 7 | | 12.86 | 4.52 |
| 10 | | 46.34 | 16.62 |

TABLE 25
GFP Expression 24 Hour After Electroporation/
Electroporation Efficiency (Data used to generate
FIGS. 11 and 12).

| Media | Donor | GFP | Avg., SD |
|---|---|---|---|
| OpT 6HDL | D032 | 49.20 | 58.99 |
| | D093 | 68.80 | (Avg.) |
| | D168 | 68.20 | 11.64 |
| | D938 | 67.75 | (SD) |
| | D242 | 41.00 | |
| OpT complete | D032 | 42.00 | 51.75 |
| | D093 | 55.80 | (Avg.) |
| | D168 | 51.92 | 5.79 |
| | D938 | 59.03 | (SD) |
| | D242 | 50.00 | |

TABLE 26
GFP Expression24 Hour After Electroporation/
Electroporation Efficiency (Data used to generate FIG. 13).

| | GFP | |
|---|---|---|
| Medium | D168 | D938 |
| OpT 6HDL | 67.00 | 64.50 |
| OpT w/o ICSR + HDL 5 + LDL 1 | 64.00 | 65.50 |
| OpT w/o ICSR + HDL 4 + LDL 2 | 53.00 | 64.00 |
| OpT w/o ICSR + HDL 3 + LDL 3 | 46.00 | 57.00 |
| OpT w/o ICSR + HDL 2 + LDL 4 | 50.00 | 56.00 |
| OpT w/o ICSR + HDL 1 + LDL 5 | 51.00 | 51.00 |
| OpT w/o ICSR + LDL 6 mg/L | 50.00 | 52.50 |
| OpT w/o ICSR | 58.00 | 52.00 |
| OpT complete | 48.00 | 50.00 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. The disclosures of which are specifically incorporated by referenced herein in their entirety.

While this invention has been particularly shown and described with references to preferred embodiments thereof, it will be understood by those skilled in the art that various changes in form and details may be made therein without departing from the scope of the invention encompassed by the appended claims.

1. A method for preparing a serum free cell culture medium, the method comprising adding a lipoprotein particle composition or a lipoprotein composition to a basal culture medium, wherein the lipoprotein particle composition or a lipoprotein composition is added in an amount to function as a serum replacement.

2. (canceled)

3. The method of claim 1, wherein the lipoprotein particle composition comprises lipoprotein particles obtained from human blood.

4.-10. (canceled)

11. A serum free cell culture medium comprising one or more lipoprotein compound made by the method of claim 1, wherein the serum free cell culture medium supports the expansion of mammalian cells and wherein the expansion of the mammalian cells is increased by at least 10% in the serum free cell culture medium comprising the one or more lipoprotein compound as compared to the same cell expanded in culture medium without the one or more lipoprotein compound but containing serum.

12.-13. (canceled)

14. The serum free cell culture medium of claim 11, wherein at least one of the one or more lipoprotein compound is a component of a lipoprotein particle.

15.-18. (canceled)

19. The serum free cell culture medium of claim 11, wherein the increase in cell viability is in the range of from 10% to about 75%.

20.-21. (canceled)

22. The serum free cell culture medium of claim 11, wherein the mammalian cells are immune cells.

23.-25. (canceled)

26. A method for expanding a mammalian cell, the method comprising incubating the mammalian cell in a serum free cell culture medium comprising one or more lipoprotein compound made by the method of claim 1 under conditions that allow for expansion of the mammalian cell.

27. The method of claim 26, wherein the lipoprotein compound comprises one or more lipoprotein particle.

28. (canceled)

29. A method for electroporation of a mammalian cell population, the method comprising:
(a) contacting the mammalian cell population with one or more lipoprotein compound for at least 12 hours in a serum free culture medium under conditions that allow for expansion of the mammalian cells, and
(b) applying one or more electric pulse to the mammalian cell population to thereby electroporate cell membranes of members of the mammalian cell population,
wherein the electroporation efficiency is at least 60% and wherein the viability of the cells in the mammalian cell population decreases by less than 10%.

30. The method of claim 29, wherein the electroporation efficiency is measured by expression of a detectable marker in members of the mammalian cell population.

31. The method of claim 30, wherein the detectable marker is a fluorescent protein.

32. A method for the maintenance of an activated T cell population, the method comprising:
(a) generating the activated population of T cells,
(b) expanding the activated population of T cells generated in step (a) in the presence of a lipoprotein supplement,
(c) exposing the expanded activated population of T cells produced in step (b) to an electric field of sufficient strength to result in a decrease in the rate of cell expansion over the following seven day by at least 30%, and
(d) maintaining the activated population of T cells of step (c) under the same conditions as in step (b) for seven days,
wherein the viability of the activated population of T cells during steps (a)-(d) remains above 70%.

33. The method of claim 32, wherein one or more nucleic acid molecule is introduced in step (c) into individual T cells of the activated population of T cells.

34.-35. (canceled)

36. The method of claim 32, wherein the activated population of T cells is expanded for three days in step (b).

37. The method of claim 32, further comprising:
(e) washing of the activated population of T cells after step (d), and
(f) expanding the washed, activated population of T cells generated in step (e) in the absence of a lipoprotein supplement.

38. (canceled)

39. The method of claim 32, wherein the activated population of T cells are shipped to a different location during step (d).

40. (canceled)

41. A method for storing mammalian cells, the method comprising the following steps in order:
(a) expanding the mammalian cells in a culture medium comprising one or more lipoprotein compound,
(b) exposing the mammalian cells to an electric field, and
(c) expanding the mammalian cells in a culture medium comprising one or more lipoprotein compound,
wherein the mammalian cells in step (c) expand at a rate that is at least 50% lower than in step (a), and
wherein the viability of the mammalian cells remains above 70% during steps (a)-(c).

42. The method of claim 41, wherein the mammalian cells are T cells.

43. The method of claim 41, wherein the mammalian cells are expanded for seven days in step (c).

44.-45. (canceled)

46. The method of claim 41, wherein a nucleic acid molecule is introduced into the mammalian cells in step (b).

47.-48. (canceled)
